(19) United States
(12) Patent Application Publication
(10) Pub. No.: US 2023/0395256 A1
(43) Pub. Date: Dec. 7, 2023

(54) PATIENT-SPECIFIC THERAPEUTIC PREDICTIONS THROUGH ANALYSIS OF FREE TEXT AND STRUCTURED PATIENT RECORDS

(71) Applicant: MEMORIAL SLOAN KETTERING CANCER CENTER, New York, NY (US)

(72) Inventors: Jacob L. Glass, New York, NY (US); Katya AHR, Philadelphia, PA (US); Deepika DILIP, New York, NY (US); Mindy KRESCH, New York, NY (US); Ross LEVINE, New York, NY (US); John PHILIP, New York, NY (US); Julie GARCIA, New York, NY (US)

(21) Appl. No.: 18/250,607

(22) PCT Filed: Oct. 26, 2021
(86) PCT No.: PCT/US2021/056687

§ 371 Date: Apr. 26, 2023

(51) Int. Cl. G16H 50/20; G16H 10/60; G16H 20/10; G16H 15/00

(52) U.S. Cl. CPC G16H 50/20; G16H 10/60; G16H 20/10; G16H 15/00

(57) ABSTRACT

Disclosed are systems and methods for retrieving, for a patient with a medical condition, a structured dataset and an unstructured dataset, the structured dataset comprising demographic and clinical data, and the unstructured dataset comprising a report with free-form text of a clinician with respect to a medical procedure (e.g., a test). Analysis may comprise applying natural language processing to the free-form text in the report to generate a plurality of health indicators for the patient. Categorizations corresponding to the medical condition may be generated, and a treatment regimen determined based on drug orders in the structured dataset. Survival modeling may be applied to generate a prediction corresponding to a survival of the patient following administration of a treatment to the patient for the medical condition. A treatment may be selected and administered based on the prediction.

### CROSS-REFERENCE TO RELATED APPLICATIONS

This application is related to U.S. Provisional Patent Application No. 63/106,206 filed Oct. 27, 2020, the entirety of which is incorporated herein by reference.

### STATEMENT REGARDING FEDERALLY SPONSORED RESEARCH

This invention was made with government support under grant number 5K08CA230172 awarded by the National Institutes of Health. The government has certain rights in the invention.

### FIELD OF THE DISCLOSURE

This disclosure relates generally to analysis of free-form text and structured patient records, and to using artificial intelligence to forecast a response of a patient to a therapy for a medical condition so as to enhance outcomes for patients.

### BACKGROUND

Determining whether a particular treatment protocol (e.g., chemotherapy) is likely to be effective generally does not take into account all available information about a patient. It also does not generally consider available data on other patients in determining likelihood of a particular patient surviving following a particular treatment. Clinicians lack the time and capacity to make sense of and consider the data that is available in electronic health records, and thus an approach that provides clinically meaningful data in a short period of time (thereby reducing disease progression), that is informed by data that would otherwise not be taken into account, would provide clinicians a valuable tool with which to significantly enhance outcomes for patients under the care of clinicians.

### SUMMARY

In various embodiments, disclosed herein is a method comprising: retrieving, by a computing system comprising one or more processors and a memory with instructions executable by the one or more processors, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure (e.g., a genetic, molecular, cellular, or chromosomal test, a radiological image, a biopsy, etc.); analyzing, by the computing system, the structured dataset and the unstructured dataset to generate a plurality of health indicators for the patient, wherein analyzing the structured dataset and the unstructured dataset comprises applying natural language processing to the free-form text in the report to extract one or more of the plurality of indicators; generating, by the computing system, based on the plurality of health indicators, one or more categorizations corresponding to the medical condition; determining, by the computing system, a treatment regimen based on drug orders in the structured dataset; performing, by the computing system, survival modeling to generate, by the computing system, based on (i) the plurality of health indicators, (ii) the one or more categorizations, and (iii) the treatment regimen, a prediction corresponding to a survival of the patient following administration of a treatment to the patient for the medical condition; and providing, by the computing system, a report comprising the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

In various embodiments, the method further comprises administering the treatment to the patient. In various embodiments, the treatment may be administered only if the prediction indicates a likelihood of survival exceeding a threshold (e.g., a prediction of at least “good” or “intermediate” risk level).

In various embodiments, the method further comprises determining that the prediction indicates a likelihood of survival exceeding a threshold. In various embodiments, the report comprises an indication of the likelihood of survival.

In various embodiments, applying natural language processing to the free-form text comprises parsing the report using a plurality of expression patterns, each expression pattern comprising one or more operators. In various embodiments, one or more of the plurality of health indicators requires one or more of the expression patterns to be triggered (matched).

In various embodiments, the one or more categorizations comprise at least one of a cytogenetic category, a radiographic category, a molecular category, or a histological category.

In various embodiments, the demographic and clinical data identifies a plurality of patient age, patient gender, the medical condition, or drugs administered to the patient.

In various embodiments, the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

In various embodiments, analyzing the structured dataset and the unstructured dataset further comprises generating tab-delimited tables based on the structured dataset.

In various embodiments, generating the tab-delimited tables comprises extracting data from unmerged nested cells and reformatting tabs into the tab-delimited tables.

In various embodiments, the medical condition is a cancer, and wherein the treatment is a cancer treatment.

Various embodiments relate to a computing system comprising one or more processors and a memory with instructions configured to be executable by the one or more processors to cause the one or more processors to: retrieve, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure; analyze the structured dataset and the unstructured dataset to generate a plurality of health indicators for the patient, wherein analyzing the structured dataset and the unstructured dataset comprises applying natural language processing to the free-form text in the report to extract one or more of the plurality of indicators; generate, based on the plurality of health indicators, one or more categorizations corresponding to the medical condition; perform survival modeling to generate, based on the plurality of health indicators and the one or more categorizations, a prediction corresponding to a survival of the patient following administration of a treatment to the patient for the medical condition; and provide a report comprising one or more categorizations and/or the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

In various embodiments, the instructions further cause the one or more processors to determine that the prediction indicates a likelihood of survival exceeding a threshold. In various embodiments, the report further includes an indication of the likelihood of survival.

In various embodiments, applying natural language processing to the free-form text comprises parsing the report using a plurality of expression patterns, each expression pattern comprising one or more operators, wherein one or more of the plurality of health indicators requires one or more of the expression patterns to be triggered.

In various embodiments, the one or more categorizations comprise at least one of a cytogenetic category, a radiographic category, a molecular category, or a histological category.

In various embodiments, the demographic and clinical data identifies a plurality of patient age, patient gender, the medical condition, or drugs administered to the patient.

In various embodiments, the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

In various embodiments, analyzing the structured dataset and the unstructured dataset further comprises generating tab-delimited tables based on the structured dataset.

In various embodiments, generating the tab-delimited tables comprises extracting data from unmerged nested cells and reformatting tabs into the tab-delimited tables.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1: Example system for implementing disclosed approach, according to various potential embodiments.

FIG. 2: Example process for predicting whether a therapy will be effective in treating a medical condition of a particular patient, according to various potential embodiments.

FIG. 3: Generalized process illustrating use of various raw structured and unstructured data to obtain various extracted and derived data, according to various potential embodiments.

FIG. 4: AML-related process illustrating use of various raw structured and unstructured data to obtain various extracted and derived data, including AML risk, according to various potential embodiments.

FIG. 5: Example analysis of cytogenetic report to determine risk category, according to various potential embodiments.

FIGS. 6A-6G: Example expression patterns and consequence of matching thereof, according to various potential embodiments.

FIGS. 7A-7C: Example diagnostic molecular pathology (DMP) report for next generation sequencing (NGS), according to various potential embodiments.

FIGS. 8A and 8B: Example chemotherapy structured (raw) data, according to various potential embodiments.

FIGS. 9A-9D: Example regimens which may be derived from extracted chemotherapy data, according to various potential embodiments.

FIG. 10: Internal and external pathology report frequency over time, according to various potential embodiments.

FIG. 11: Frequency of different pathology report types, according to various potential embodiments.

FIG. 12: Frequency of different ELN clinical risk categories, according to various potential embodiments.

FIG. 13: Oncoprint of mutations associated with cytogenetic and ELN risk categories, according to various potential embodiments.

FIG. 14: Clinical risk associated with common cytogenetic and molecular categories, according to various potential embodiments.

FIG. 15: Influence of FLT3-ITD quantitative level on overall survival, according to various potential embodiments.

FIG. 16: Treatment regimens used in de-novo and relapsed disease, according to various potential embodiments.

FIG. 17: Treatment regimens stratified by patient age, according to various potential embodiments.

FIG. 18: A simplified block diagram of a representative server system and client computer system usable to implement certain embodiments of the present disclosure.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

Modern disease diagnosis and treatment can be highly data-driven. Each leukemia assessment may involve, for example, staining slides with multiple antibodies, performing multidimensional flow cytometry, cytogenetic assessment including karyotype, fluorescence in-situ hybridization (FISH), and/or single nucleotide polymorphism (SNP) arrays, next generation sequencing (NGS) testing for tens to hundreds of gene mutations and/or rearrangements, and targeted molecular assays. Data from such studies are interpreted by hematopathologists and summary reports are deposited in the electronic medical record (EMR) alongside physician notes, other lab results, and treatment data. Various embodiments employ a natural language processing (NLP) based system to extract relevant data from these reports, process the findings to provide automated risk stratification and treatment regimen information, and provide tools to rapidly perform retrospective clinical studies and share these results.

Retrospective clinical studies have long been collaborative efforts between senior academic clinicians and trainees. To test a hypothesis the senior clinician has, a trainee will often review thousands of pages of test results, documentation, and drug orders to generate a dataset capable of testing that hypothesis. This process of manual curation is frequently very time consuming, involving weeks to months of manual review of EMR data followed by entry in a spreadsheet. The spreadsheet data is then shared with a statistician who will assist in formal hypothesis testing. In the event that this leads to additional questions, additional data may need to be gathered, resulting in another round of manual curation and data entry.

Following completion of a study, these spreadsheets are often filed away and frequently are forgotten or misplaced once the trainee moves on. In addition, formatting and variable labeling are generally ad hoc, making it difficult to adapt a spreadsheet from one study to the next.

Various embodiments of the disclosed approach shorten this duration of curation from months to minutes, unlocking the data that is already stored electronically in the EHR, and processing it to generate clinically meaningful information such as disease risk or treatment regimen immediately available. In addition, the system is designed in a modular fashion, making the process of updating clinical guidelines and treatment regimens simple.

In addition, various embodiments simplify processed data storage and retrieval. Instead of stored spreadsheets, processed and generated data may be stored in a central database, with each feature identified by a universal concept ID. In addition, through an “Extract” system these studies may be accessible to other users through a system that involves an online data shopping cart and is organized according to the data generator's sharing parameters and governed by Institutional Review Board (IRB) guidelines.

### Overview of Systems and Methods

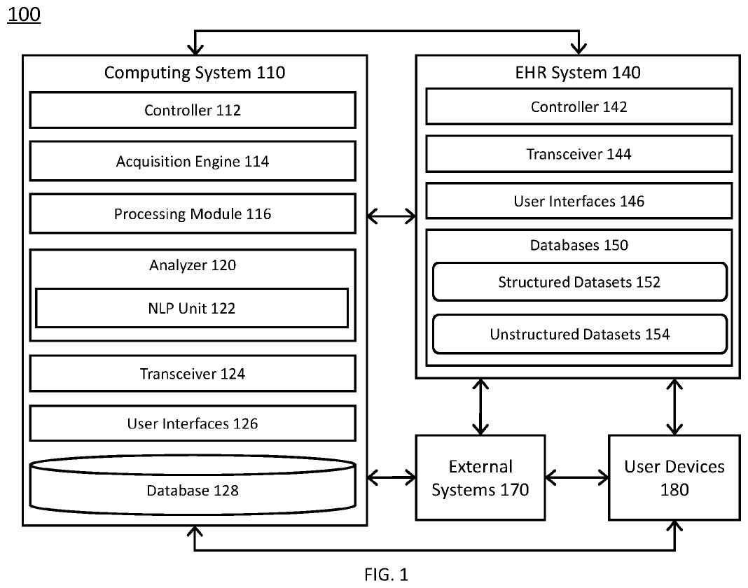

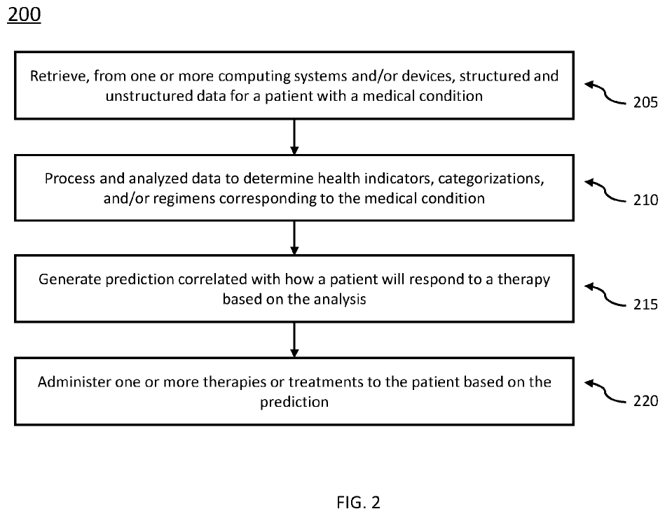

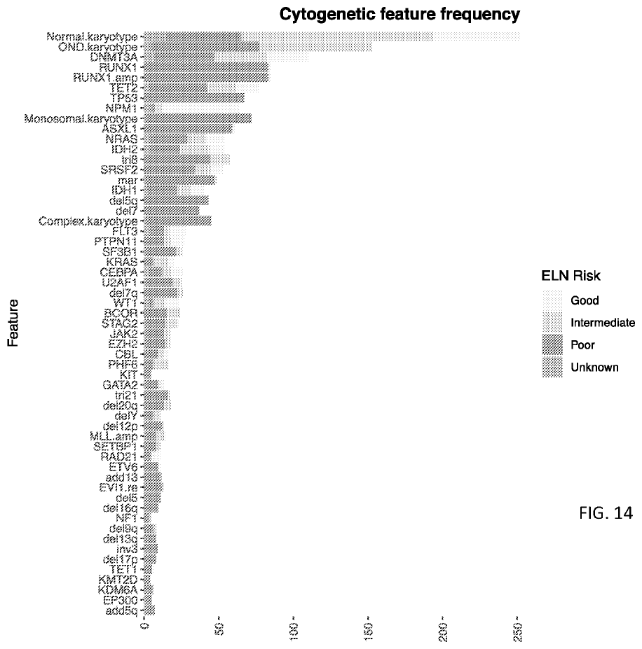

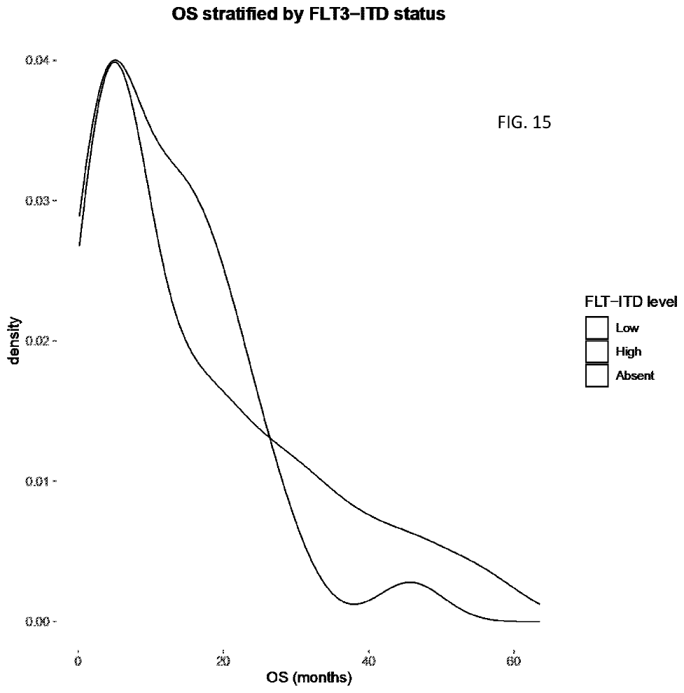

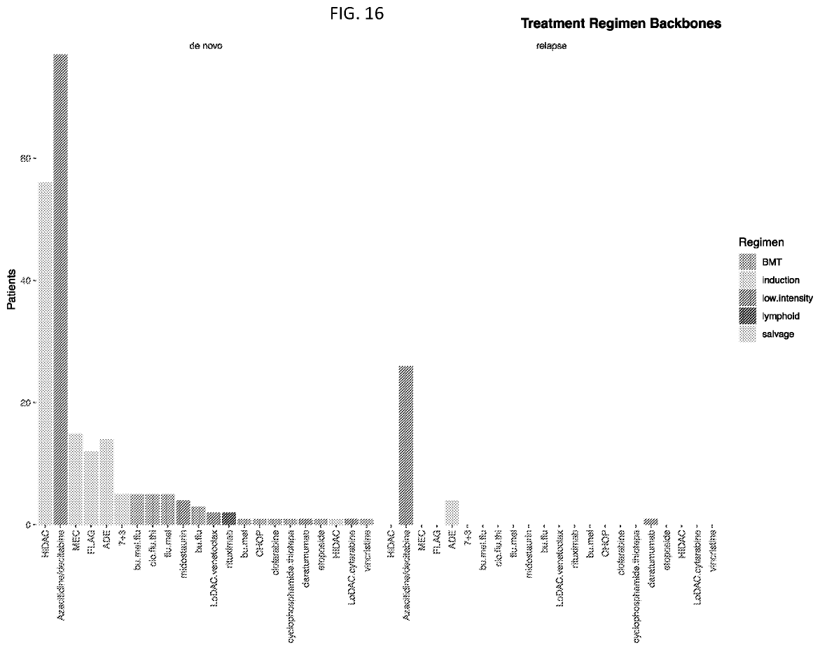

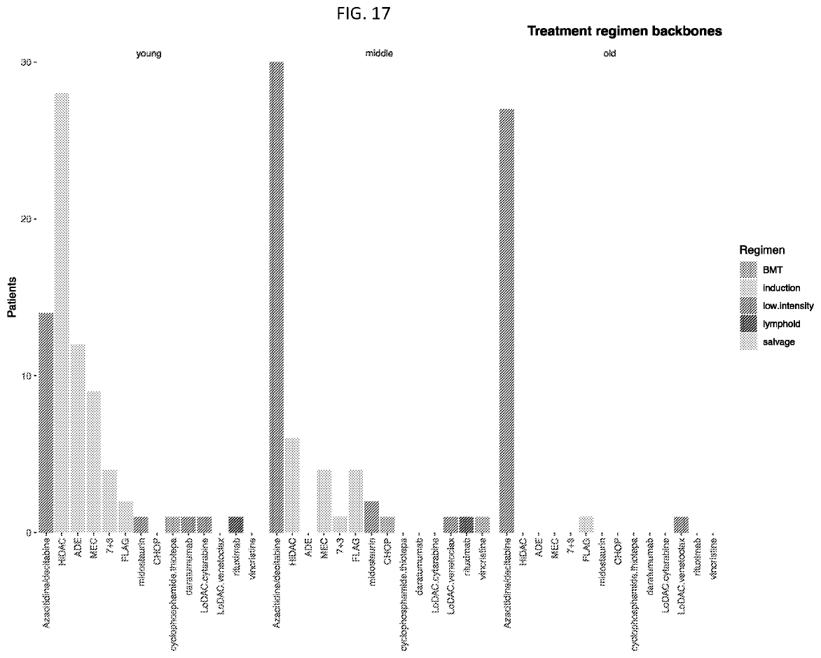

Referring initially to FIG. 1, in various embodiments, a system 100 may be used to implement example process 200 (see FIG. 2) and the overall approach disclosed herein. The system 100 may include a computing system 110 (which may be one or more than one computing devices, co-located or remote to each other), an electronic health record (EHR) system 140, one or more external systems 170, and one or more user devices 180. The external systems 170 may include, for example, systems of other institutions and/or other sources of patient-specific or general health data. User devices 180 may include devices of clinicians, researchers, or others providing or receiving data on specific patients. In various implementations, the computing system 110 and the EHR system 140 may be integrated into one system, or may be separate and distinct systems in communication with each other over a communications network. In certain implementations, computing system 110 (or components thereof) may include one or more user devices 180. In various potential setups, with reference to FIG. 14, the EHR system 140 may correspond to a server system 1800 with respect to the computing system 110 and/or the user devices 180 serving as client computing systems 1814. Similarly, the computing system 110 may serve as a server system 1800 with respect to user computing devices 180 serving as client computing systems 1814 that send and/or receive patient data. Additionally, each external system 170 may serve as a server system 1800 with respect to the computing system 110, the EHR system 140, and/or the user devices 180 serving as client computing systems 1814.

The computing system 110 (with one or more multiple computing devices) may be used to retrieve data from or via, directly or indirectly, EHR system 140, one or more external systems 170, and/or one or more user devices 180. The computing system 110 may include one or more processors and one or more volatile and non-volatile memories for storing computing code and data that are captured, acquired, recorded, and/or generated. The computing system 110 may include a controller 112 that is configured to exchange signals and data with EHR system 140, external systems 170, and/or user devices 180, allowing the computing system 110 to be used to obtain data to be analyzed and/or provide results of various processes and analyses. The computing system 110 may include an acquisition engine 114 configured to obtain patient data, a processing module 116 configured to pre-process data, an analyzer 120 configured to analyze data from acquisition engine 114 and/or processing module 116. The analyzer 120 may include a natural language processing (NLP) unit 122 configured to perform natural language processing or other artificial intelligence techniques on patient data. In various embodiments, analyzer 120 may also include a karyotype parser (not pictured) configured to extract karyotypes from reports, as further discussed below. In certain embodiments, NLP unit 122 may also serve as, or perform functions of, a karyotype parser.

A transceiver 124 allows the computing system 110 to exchange data, wirelessly or via wires, with EHR system 140, external systems 170, and/or user devices 180. One or more user interfaces 126 allow the computing system to receive user inputs (e.g., via a keyboard, touchscreen, microphone, camera, etc.) and provide outputs (e.g., via a display screen, audio speakers, etc.). The computing system 110 may additionally include one or more databases 128 for storing, for example, raw and processed patient data and results of analyses. In some implementations, database 128 (or portions thereof) may alternatively or additionally be part of another computing device that is co-located or remote and in communication with computing system 110.

EHR system 140 may additionally databases 150, which comprise structured datasets 152 and unstructured datasets 154. Structured data may in a standardized format, providing information with classifications, categorizations, or labels that define its content. Structured data may be highly organized and more readily decipherable. For example, the organized and predefined architecture of structured data may make it more easily usable by machine learning algorithms. However, their relative ease of use and accessibility comes at the cost of inflexibility. Unstructured data, by contrast, may include data that is not readily analyzable using conventional tools and methods. Because unstructured data does not impose a specific, predefined data architecture, it is more flexible and versatile, and increases the pool of available data because predefined formats, labels, rules, etc., are not necessarily required. Due to its relatively undefined nature, however, processing and analyzing unstructured data involves unique data science expertise and specialized tools. EHR system 140 may also include a controller 142, a transceiver 144, and user interfaces 146 analogous to controller 112, transceiver 124, and user interfaces 126, respectively.

External systems 170, which may also include controllers, transceivers, user interfaces, and databases, may be computing systems of other institutions, other EHR systems, or other networked sources of data. Examples of user devices 180 may include smartphones, tablet computers, laptops, desktop computers, workstations, wearable smart devices, vehicles, Internet of Things (IoT) or other smart devices, and/or other computing devices that can collect and/or present raw or processed data and analyses thereof.

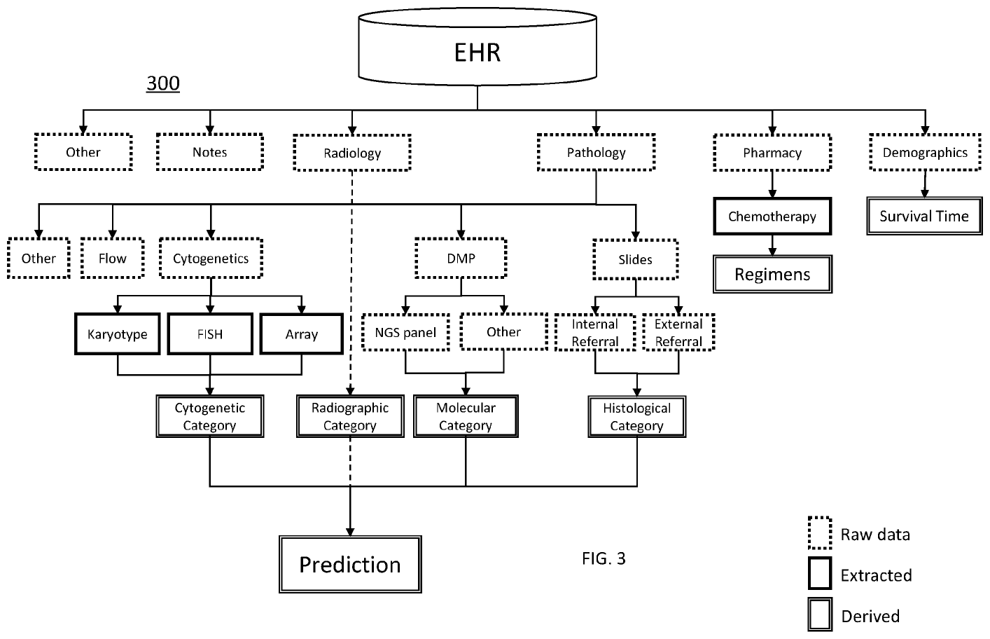

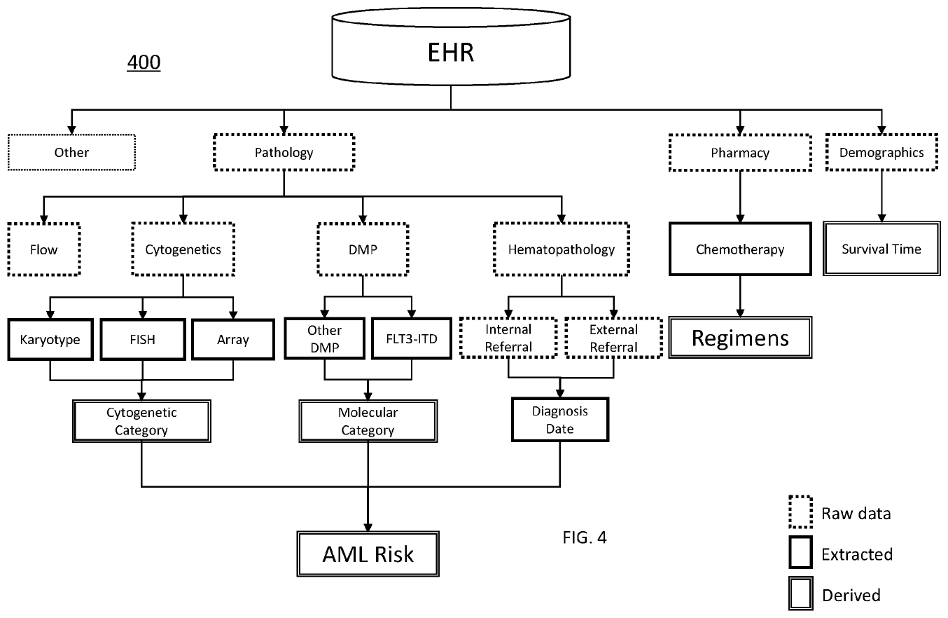

With reference to FIG. 2, an example treatment evaluation process 200 is illustrated, according to various potential embodiments. Process 200 may be implemented by or via one or more computing devices of computing system 110. At 205, structured and unstructured datasets for a patient with a medical condition may be retrieved. In various embodiments, the computing system 110 may (e.g., via acquisition engine 114) receive such data from EHR system 140 (e.g., data in databases 150), external systems 170, and/or user devices 180. As further discussed with respect to FIGS. 3 and 4, examples of structured data include data on demographics of the patient (e.g., age, gender, race, etc.), test results (e.g., genetic tests such as diagnostic molecular pathology (DMP), flow cytometry, and/or hematopathology), internal and external patient referrals, pharmaceutical orders (e.g., chemotherapeutics or other drugs), etc. Examples of unstructured data include free-form text or other prose, such as discussion of test results and recommendations for next steps, or other notes by clinicians. Such free-form text may relate to, for example, a pathology report or a report discussing findings of radiological imaging.

At 210, the raw data obtained at 205 may be processed (e.g., by processing module 116) and analyzed (e.g., by analyzer 120) to extract health indicators (related to, e.g., karyotype, FISH, SNP array, genetic tests such as DMP, FLT3-ITD, chemotherapy, and diagnosis dates) and derive categorizations (e.g., a cytogenetic category, a radiographic category, a molecular category, a histological category, a treatment regimen, and/or survival time). At 215, the health indicators, categorizations, and/or regimens are used to generate a prediction of how a patient is expected to respond to a treatment or therapy for the medical condition. This prediction (e.g., cancer risk, such as risk of acute myeloid leukemia (AML)) is a prognostic estimate of how a patient will respond to the treatment or therapy (e.g., traditional chemotherapy). A prediction of “good” may mean a good chance of responding to the treatment or therapy (e.g., a good chance the patient can be cured with chemotherapy alone), while “intermediate” or “poor” risk patients may be recommended to have a second treatment or therapy (e.g., a bone marrow transplant following chemotherapy may be warranted to cure the patient of the medical condition). As example estimates, most (e.g., about 60%) of good risk patients may be cured, while fewer intermediate risk patients (e.g., 40% to 50%) may be cured, and fewer still (e.g., about 20%) of poor risk patients may be expected to be cured by the treatment or therapy. At 220, one or more therapies or treatments (e.g., medicines, surgical procedures, etc.) may be administered to the patient based on the prediction.

Referring to FIGS. 3 and 4, a generalized process 300 and an AML-specific example process 400 illustrate using various raw structured and unstructured data to obtain various extracted and derived data. A computing system (e.g., computing system 110) may obtain (e.g., from EHR system 140) raw data (as indicated by the dotted boxes) related to demographics (e.g., age, gender, race), pharmacy (e.g., medicines administered), pathology (e.g., medical conditions), radiology (e.g., images taken), notes (e.g., reports on pathological and radiological tests or images), and tests and assays (e.g., flow cytometry, cytogenetic assessment, fluorescence in-situ hybridization (FISH), a single nucleotide polymorphism (SNP) array, next generation sequencing (NGS) panels, DMP, slides, etc.), internal referrals (e.g., a referral for specialized care from a clinician at the institution or facility associated with the computing system 110 at which the patient is being treated for the medical condition) and external referrals (e.g., at another institution or facility which may have diagnosed or administered treatments to the patient for the medical condition, such as an institution or facility associated with an external system 170). The raw data may be used to extract certain data (as indicated by the single solid line boxes) such as karyotype, FISH, SNP array, FLT3-ITD, chemotherapy, and diagnosis date. The extracted data may be analyzed to derive certain other data (as indicated by the double solid line boxes) such as cytogenetic category, radiographic category, molecular category, histological category, regimens, survival time, and a survival prediction (such as AML risk in the case of AML).

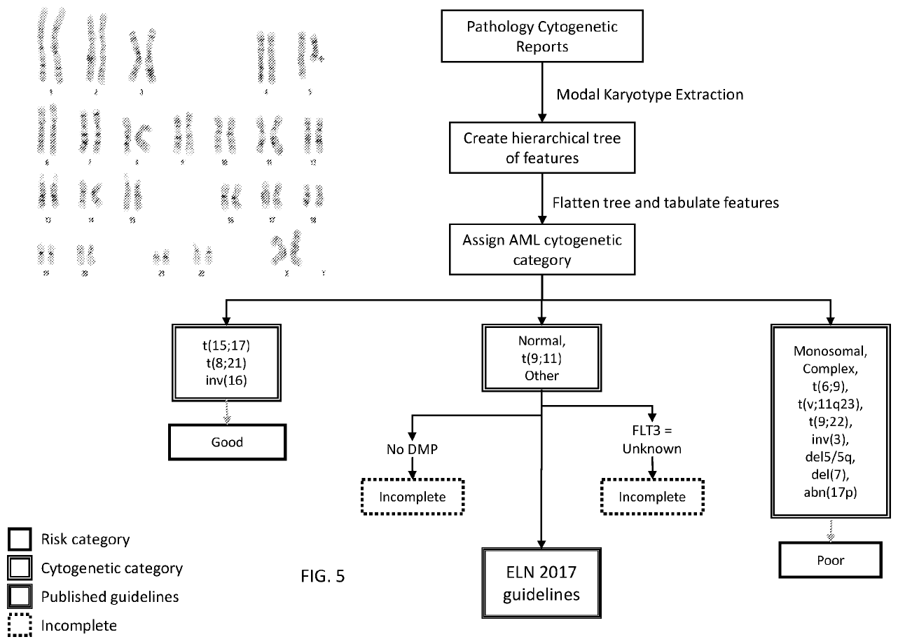

Referring also to FIG. 5, and the below example pathology cytogenetic report, the modal karyotype is obtained from the primary source of cytogenetic data. The cytogenetic report also provides an example karyotype description and FISH description, both of which can be processed using the expression patterns disclosed herein.

In various embodiments, the karyotype extracted from the modal karyotype line (by, e.g., a karyotype parser) is in ISCN format (International System for Human Cytogenetic Nomenclature). The karyotype parser may identify each feature described in the modal karyotype line, the parent clone, and the number of cells seen with that pattern. For example 46,X,Y,t(3;3)(q21;q26.2) [14]/idem,del7(q22q34)[4], idem, add(17)(p12)[2] means that there is a normal male karyotype with an inversion on chromosome 3 in 14 cells and a subclone (idem) with that pattern plus a partial deletion of chromosome 7 in 4 cells, and a separate subclone with an additional chromosome 17 in 2 cells. The parser may then simplify that hierarchical data structure into a table for AML risk analysis (but the original data could be used for other research purposes too). An example hierarchical tree may be as follows:
Parent 1: original text: “46,X,Y,t(3;3)(q21;26.2)[14]”, #chromosomes: 46, sex chromosomes: XY, feature 1:translocation, source: chromosome 3, subfeature: location=q21, target: chromosome 3, subfeature: location=q26.2, cells=14
Child 1: parent=parent 1, original text: “idem, de17(q22q34)[4]”, feature 1: deletion, location1: q22, location 2: q34, cells=4
Child 2: parent=parent 1, original text: “idem, add(17)(p12)[2]”, feature 1: addition, location: p12, cells=2

An example of how this tree may be flattened is a table as follows:
Chromosomes #, t(3;3), de17(q), add(17), other.1, other.2
46, True, True, True, False, . . .
Where other.1, other.2, etc. are other translocations for other patients in the dataset.
Also, features with low cell counts—for example, a cell count of 1—may be excluded from the table.

### Example Methods

It is noted that although example methods are discussed with respect to AML and treatment and survivability thereof, the disclosed approach is applicable to other medical conditions (cancer and non-cancer). Various embodiments may employ a software package in R to extract information from the electronic medical record (EMR, used interchangeably with EHR) including diagnosis, cytogenetic, and molecular characteristics. Hematopathology, flow cytometry, cytogenetic, and diagnostic molecular pathology (DMP) reports available in the EMR were reviewed for bone marrow and peripheral blood evaluations that occurred during the period of follow-up.

Pre-Processing: Data queries from an institutional database may return an Excel spreadsheet. Various embodiments may employ a series of functions that extract the data from unmerged nested cells and reformat tabs into individual, tab-delimited tables. Embodiments may identify columns with dates and use the UTC time zone to standardize them.

To begin text extraction, a set of functions may be employed to acquire dates of diagnosis. An example embodiment first uses the current time as input to get the origin date, convert that date to integer format, convert the integer format back to date format, and consolidate overlapping date ranges into a data table. Next, a subset of data closest to the dates, after the dates, and before the dates in this data table are captured. Dates by or near overlaps are then consolidated.

Data Parsing: Demographic data from patients may be incorporated for purposes of stratification by age and determining survival probabilities based on mutations and cytogenetic abnormalities.

Parsing Pathology Reports

Hematopathology: In an example embodiment, internal hematopathology reports are identified using report headers. The text of these reports are then cleaned for formatting irregularities and common spelling mistakes, and split into paragraph blocks. The diagnostic summary paragraph is identified and then compared to a regular expression consisting of an exhaustive set of patterns consistent with a diagnosis (e.g., of Acute Myeloid Leukemia (AML) or High Grade Myeloid Neoplasm (HGMN)) using non-greedy matching. Reports matching these diagnoses are flagged and added to a table including the diagnostic text, date of procedure, material source (e.g., bone marrow or blood) and original full length pathology report.

In an example embodiment, hematopathology reports resulting from external referrals in which bone marrow slides and/or material from an outside institution are reviewed are processed in a similar manner. The main difference lies in identification of the procedure date, which is extracted from a different location in the text report.

In an example embodiment, dates of diagnosis may be assigned based on the procedure date of the first bone marrow biopsy showing a positive result for AML or HGMN (or other medical condition). Internal and external results are merged, allowing for diagnoses to be made from dates earlier than arrival at the current institution if material was reviewed from an earlier timepoint.

Cytogenetics: Similar to identification of hematopathology reports, in an example embodiment, cytogenetics reports are identified on the basis of the report headers. Diagnostic text is extracted in a similar fashion, but also allows for reports containing multiple or no diagnoses. The diagnostic interpretation of cytogenetics is then split into separate karyotype and FISH components. A helper function processes cytogenetic pathology reports into a data table by extracted diagnosis, and updates cytogenetics using priority vectors. Cytogenetic features are then assigned based on pathology.

Additionally, an example embodiment uses a parser-approach in which the modal karyotypes from pathology reports are split and formulated into a feature hierarchical tree. The clones may be aggregated into a tabular format. Clones with cell counts of 1 were not included for the purposes of assigning cytogenetic categories. The karyotype was subsequently assigned a cytogenetic category of complex, monosomal, CBF (core-binding factor), normal, or other-not-determined abnormalities. Karyotypes with insufficient cell counts were designated as incomplete for both cytogenetics and AML risk (or other prediction). Sensitivity and specificity metrics may be reported using the parser due to increased accuracy.

Diagnostic Molecular Pathology

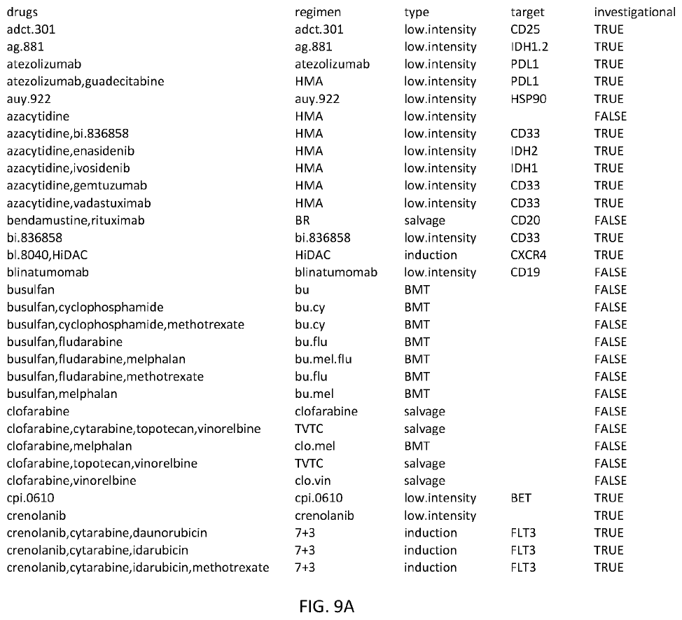

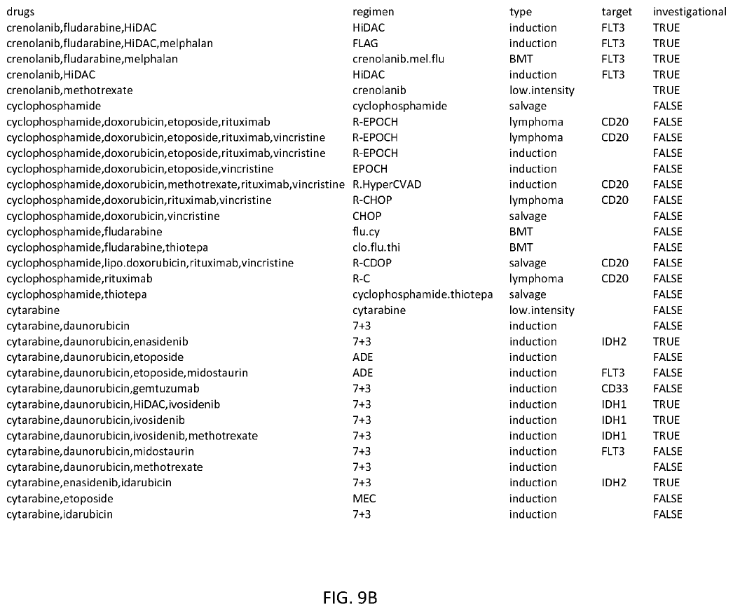

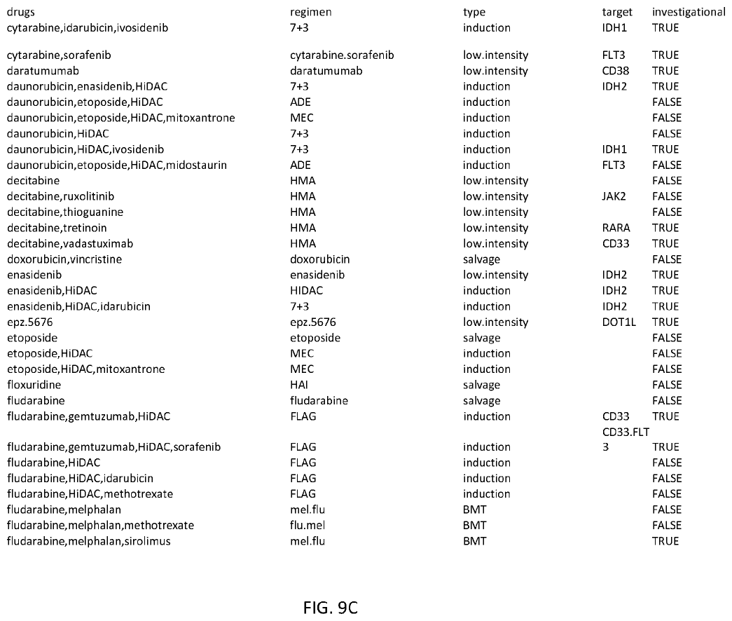

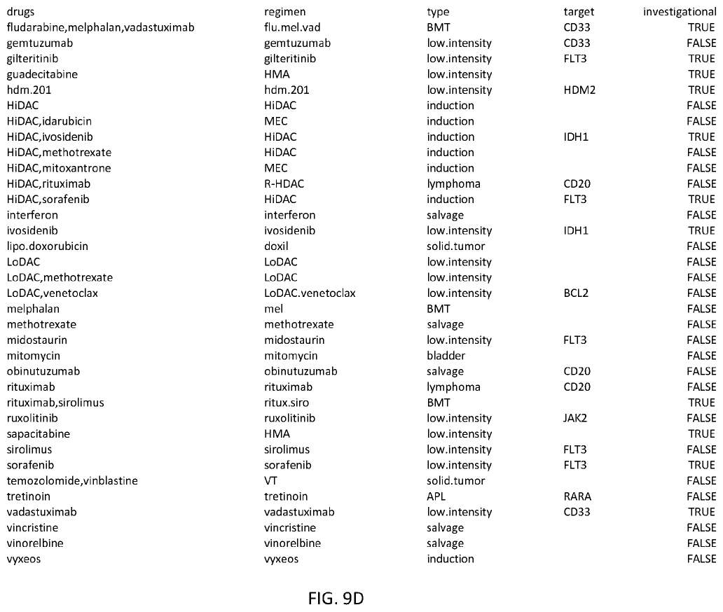

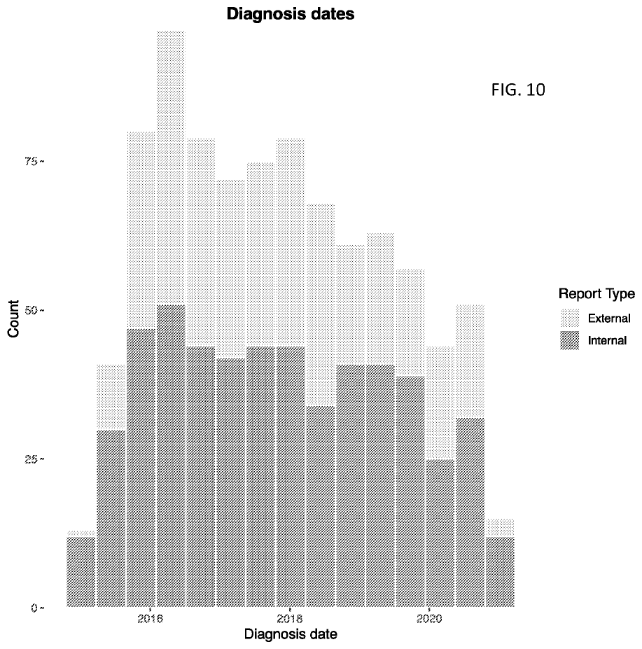

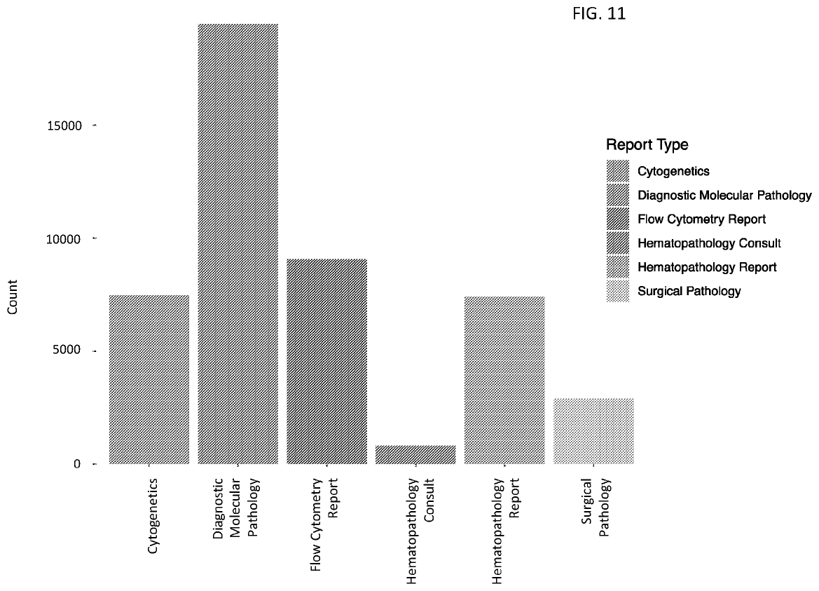

In various embodiments, to analyze diagnostic molecular pathology (DMP) reports, the program may first load structured mutation reports from the corresponding file and convert dates to POSIX (Portable Operating System Interface) calendar format using the UTC time zone. Mutation features and variant allele frequencies (VAFs) are loaded into a variant table, then the long format variant table is converted to wide with VAF as entry and NA for empty cells. Bi-allelic CEBPA (CCAAT Enhancer Binding Protein Alpha) mutations are identified in which mutations at two distinct loci in the CEBPA gene exist at the same timepoint, and a dedicated column is added to the table. Likewise, dedicated quantitative capillary-based FLT3 testing is parsed from a separate report and added to the table. Three versions of the table are generated in a list: one with quantitative VAFs for each feature, one with Boolean True/False values for each feature, and a third with semicolon separated gene mutation information suitable for oncoprint generation (see FIG. 9 for an example oncoprint).

Flow Cytometry

In various embodiments, a series of regular expressions are used to identify flow cytometry findings such as abnormal myeloid or abnormal B-cell populations. In addition, using a finite state machine based approach, individual flow cytometry markers such as CD34 or CD19 are tabulated using the information provided in the report.

For these patients, various embodiments of the disclosed pipeline may be employed to extract and tabulate cytogenetic data from cytogenetic reports corresponding to their dates of diagnosis. Molecular and cytogenetic data may be subsequently merged and processed to assign AML risk according to current European Leukemia Net (ELN) guidelines.

Blast Percentage

Hematopathology reports often include a quantitative estimate of disease burden in the form of a blast percentage. These may be reported from an assay on the marrow aspirate, marrow biopsy, or both. Using a similar approach to identification of the diagnostic paragraph, the report section containing these estimates is identified, and the blast percentage is extracted using a custom set of regular expressions. These estimates may be quantitative (ex. “25%”) or qualitative (ex. “not increased”). Both types of data may be gathered for later use.

Risk Assignment

Clinical AML categories of ‘Good’, ‘Intermediate’, and ‘Poor’ risk may be assigned according to 2016 ELN criteria using combined cytogenetic and DMP data processed above. This assignment may be made in two passes, one for cytogenetically defined risk, and one for molecular. See example risk stratification and associated genetic abnormalities in Table 1 below.

TABLE 1
Risk stratification by genetics in non-APL (acute promyelocytic leukemia) AML

| Risk Category* | Genetic Abnormality |
|---|---|
| Favorable | t(8;21)(q22;q22.1); RUNX1-RUNX1T1 |
| | inv(16)(p13.1q22) or t(16;16)(p13.1;q22); CBFB-MYH11 |
| | Biallelic mutated CEBPA |
| | Mutated NPM1 without FLT3-ITD or with FLT3-ITD^{low†} |
| Intermediate | Mutated NPM1 and FLT3-ITD^{high†} |
| | Wild-type NPM1 without FLT3-ITD or with FLT3-ITD^{low†} (without adverse-risk |
| | genetic lesions)t(9;11)(p21.3;q23.3); MLLT3-KMT2A^{‡} |
| | Cytogenetic abnormalities not classified as favorable or adverse |
| Poor/Adverse | t(6;9)(p23;q34.1); DEK-NUP214 |
| | t(v;11q23.3); KMT2A rearranged |
| | t(9;22)(q34.1;q11.2); BCR-ABL1 |
| | inv(3)(q21.3q26.2) or t(3;3)(q21.3;q26.2); GATA2,MECOM(EV11) |
| | −5 or del(5q); −7; −17/abn(17p) |
| | Complex karyotype,§ monosomal karyotype ∥ |
| | Wild-type NPM1 and FLT3-ITD^{high†} |
| | Mutated RUNX1^{¶} |
| | Mutated ASXL1^{¶} |
| | Mutated TP53^{#} |

[symbol]

*Prognostic impact of a marker is treatment-dependent and may change with new therapies.
^{†}Low, low allelic ratio (<0.5); high, high allelic ratio (≥0.5); semiquantitative assessment of FLT3-ITD allelic ratio (using DNA fragment analysis) is determined as ratio of the area under the curve ″FLT3-ITD″ divided by area under the curve ″FLT3-wild type″; regardless of FLT3 allelic fractions, patients should be considered for HCT, though recent studies indicate that AML with NPM1 mutation and FLT3-ITD low allelic ratio may also have a more favorable prognosis and patients should not routinely be assigned to allogeneic HCT. FLT3 allelic ratio is not yet pervasively used, and IF not available, the presence of an FLT3 mutation should be considered high risk unless it occurs concurrently with an NPM1 mutation, in which case it is intermediate risk. As data emerge, this measure will evolve.
^{‡}The presence of t(9;11)(p21.3;q23.3) takes precedence over rare, concurrent adverse-risk gene mutations.
§Three or more unrelated chromosome abnormalities in the absence of 1 of the WHO-designated recurring translocations or inversions, that is, t(8;21), inv(16) or t(16;16), t(9;11), t(v;11)(v;q23.3), t(6;9), inv(3) or t(3;3); AML with BCR-ABL1.
∥ Defined by the presence of 1 single monosomy (excluding loss of X or Y) in association with at least 1 additional monosomy or structural chromosome abnormality (excluding CBF AML).
^{¶}These markers should not be used as an adverse prognostic marker if they co-occur with favorable-risk AML subtypes.
^{#}TP53 mutations are significantly associated with AML with complex and monosomal karyotype. [symbol]
indicates data missing or illegible when filed

Cytogenetic Risk

In various embodiments, adequate karyotype assessment may be defined as, for example, >=20 total cells assessed, with >=2 cells needed to define a clonal population. Non-clonal populations or those not detected by karyotype could also be determined by FISH or SNP array findings. For cases in which number of cells assessed was adequate, in an example embodiment, good cytogenetic risk was defined by t(8;21), inv(16), or t(15;17) and was assigned highest priority. The intermediate risk t(9;11) was assigned the next priority, followed by poor risk features. Any undefined abnormalities including normal karyotype were assigned the lowest priority, conferring intermediate cytogenetic risk. These risk categories are codified in the ELN cytogenetic risk table, allowing for simple updates with future ELN revisions.

Molecular Risk

In an example embodiment, molecular risk may be assessed next and allowed to confer poorer clinical risk than that dictated by cytogenetic risk, but not better, consistent with current ELN guidelines and the supporting literature. In addition, poor risk ASXL1 and RUNX1 mutations were not permitted to supersede a good risk or t(9;11) intermediate risk designation, nor were any molecular features permitted to change a cytogenetically-based good risk designation. FLT3-ITD assessment was performed quantitatively per ELN guidelines, with a VAF>=50% conferring a high risk in the absence of NPM1 mutation or intermediate risk with an NPM1 mutation in the setting of a normal karyotype. A FLT3-ITD VAF<=50% was assigned good risk if it co-occurred with an NPM1 mutation or intermediate risk without NPM1 mutation in the context of a normal karyotype. Karyotypes without a dedicated FLT3-ITD assessment with a normal karyotype were considered incomplete cases.

Chemotherapy Regimens

In addition to risk criteria, various embodiments may employ drug orders to identify the chemotherapeutic regimens each patient received. Chemotherapy routes of administration are loaded for a subset of standard drug names, dates of administration converted to POSIX calendar time format, and irrelevant routes of administration such as hepatic infusion disregarded. Chemotherapy date ranges are then consolidated by intermittent, continuous, and combined administration with the number of doses. Chemotherapy orders are then converted to treatment regimens by drug, dose, and duration, with different intensity therapies classified by their appropriate dosages.

In an example embodiment, chemotherapy orders for a set of patients were processed to standardize drug names, and filtered by administration route where available. Drugs given intrathecally were filtered out as well as standard intrathecal regimens in which administration route was not available. The remaining drugs were then separated into continuous and episodically administered agents. Episodically administered drugs were then clustered temporally, and continuous agents were added back. Drug combinations were then converted to chemotherapy regimens and appropriate metadata was added regarding drug targets, regimen intensity, and standard vs. investigational agents. Drug dosage was incorporated as appropriate—particularly in regimens using either high or low dose cytarabine.

Survival Modeling

In various embodiments, using overall survival from original date of diagnosis as an endpoint, contributions of patient age, molecular data, assigned ELN risk, and treatment regimen may be assessed using multivariate Cox Proportional Hazards modeling for good, intermediate, and poor risk patients. Significance was assessed by a Wald test of each variable within the model.

Identification of Lineage Ambiguity

In an example embodiment, a corpus was created from the free text of flow cytometry reports. After text cleanup, the example embodiment extracted the diagnostic summary paragraphs and identified the specific diagnosis using a custom set of functions. Sample acquisition and procedure dates were extracted and converted into a standard date format. The example embodiment extracted the formal diagnosis from the diagnostic summary paragraph. In cases where more than one diagnosis was suggested or an ambiguous diagnosis was noted, these findings were recorded as well. Because lineage ambiguity may evolve with treatment and become clearer with additional diagnostic and clinical data, flow reports from all available disease timepoints may be evaluated to determine the formal diagnosis. Specific abnormal lineages including B cell, T-cell, myeloid, and plasma cells were tabulated in the example embodiment.

Diagnosis of Mixed Phenotype Acute Leukemia (MPAL)

MPAL is a determined based on immunophenotype (ELN, World Health Organization (WHO) 2016) and exclusion of other diagnoses. The initial flow cytometry evaluation diagnosis is often ambiguous while other studies are being performed and additional clinical data is being gathered. Therefore, various embodiments may integrate information from hematopathology and flow cytometry reports over all available timepoints, distinguishing suggested or putative diagnoses from definitive ones. In addition to diagnosis, information regarding sample adequacy, specific abnormal lineages, and the presence or absence of specific surface markers may be extracted.

In various embodiments, to integrate various putative diagnoses and lineage information, a diagnostic rank list may be used to accurately assign diagnosis when more than one was recorded. In addition, definitive diagnoses may be prioritized over putative ones. This ranking is listed as follows: AML-MRC, CML, B/myeloid, T/myeloid, MPAL, T-ALL, B-ALL, T-ALL.ETP, t-AML, AML, leukemia, NA. After definitively confirming that a patient's diagnosis was not AML-MRC or t-AML, a second ranking may be used to accurately assign one diagnosis to each patient. This ranking is listed as follows: B/myeloid, T/myeloid, MPAL, T-ALL, B-ALL, T-ALL.ETP, CML, AML-MRC, t-AML, AML, leukemia, NA.

To incorporate immunophenotypic shifts over time, various embodiments include an additional set of sub-diagnoses. These are MPAL with simultaneous expression of multiple lineages, MPAL with sequential expression of multiple lineages, MPAL with B/myeloid immunophenotype, MPAL with T/myeloid immunophenotype, MPAL-NOS.

Survival Analysis of MPAL and AML-MRC:

Currently, there are no formal clinical guidelines for assessing clinical risk in MPAL patients. For the study of lineage infidelity in MPAL and near MPAL cases, various embodiments may apply current ELN criteria for AML to these cases given the myeloid lineage dominance in this cohort and enrichment for AML-MRC cases. Using overall survival as the endpoint, contributions of cytogenetic risk, age, and molecular data were evaluated using multivariate Cox Proportional Hazards modeling. An example embodiment used the date of the patient's first pathology report reviewed at an institution as the putative date of diagnosis. The example embodiment categorized cytogenetic risk in accordance with European LeukemiaNet 2017 (ELN) criteria for AML. 1-2 cytogenetic abnormalities were considered intermediate risk. Monosomal karyotype, complex cytogenetics, t(9;22) and MLL rearrangement were included in the high-risk category. Good risk patients were excluded from this analysis.

Data Sharing

Certain embodiments may include a Shopping Cart, a web application built with a React.js front end and a Python Aiohttp server on the back end. The Extract Datamart may be deployed on an IBM DB2 mainframe and houses the full-text reports and discretized data that is extracted by the disclosed system. In various embodiments, a Terminologist UI is a web application written in Java, with a React.js front end that provides terminology teams with the capabilities to extend REDCap metadata, build a library of standardized data elements, and standardize source metadata by mapping them to standardized Concept IDs. Concept ID service may be a Python Flask API (application programming interface) that is used to dynamically pull data from the Extract Datamart, as well as perform data governance checks to ensure no unauthorized patient data is shared.

Data Storage and Portability

Data generated by the disclosed approach may be stored in an institutional database (e.g., database 128). Although some clinicians may be granted access to this system upon request and IRB approval, few use it due to the technical expertise required to access and interpret it. Instead, in various embodiments, most clinicians may be sent the output of a specific query in spreadsheet format which they will work on locally. More recently, clinicians have begun using REDCap, a multiuser web-based electronic data capture system capable of performing HIPAA compliant surveys and/or data storage via a MySQL or MariaDB back end. This system allows for centralized long-term data storage, and data can be deposited by simply uploading the contents of a specially formatted set of spreadsheets.

To facilitate sharing and re-use of REDCap projects and other custom databases, various embodiments may employ a custom platform (e.g., Memorial Slone Kettering Extract (“MSK Extract”)). Data from all projects connected to MSK Extract are stored in a Datamart within MSKCC's institutional database. A web interface incorporating project specific permissions and IRB approval, au be built to allow users to select data from any available project using a shopping cart interface. When users check out, the data is processed through a carefully curated set of concept IDs, ensuring that elements such as ‘gender’ and ‘sex’ are mapped to the same data element. This data is then deposited in a new REDCap project and can be automatically visualized using data visualization software (e.g., Tableau by Tableau Software, LLC). In addition, programmatic access to the data may be available through the REDCap API. Users may upload data to be shared through this interface as well.

MSK Extract allows for a crowdsourcing approach to building a standard library. Names for individual data elements are still customizable unlike most other terminology approaches. The use of Concept IDs provides internal standardization, and these standard data elements can be used for other REDCap projects. Standard concepts are also the basis for an API service that delivers data automatically for REDCap projects. Data visualizations built from standard concepts allow simple and accurate visualization across multiple REDCap projects.

This system, in combination with the disclosed pipeline, allows for research teams to quickly build a database of patient data. From this REDCap database, they can then build visualizations, perform data analysis, and share data back to the greater research community without having to perform manual abstraction from clinical notes and reports. This system will save time and allow research to progress more quickly in the future.

### Results of Example Embodiments

### Data Capture

Records included free-text hematopathology reports, free text flow cytometry reports, free-text cytogenetic reports, free-text and structured molecular diagnostic reports, tabulated drug administration records, tabulated complete blood count records, and demographic data. Cytogenetic data included karyotypes, FISH, and SNP arrays. Mutation data from 2015 and onward was tabulated based on structured data in long format produced from next generation sequencing mutation studies. These data may also be extracted from free text reports. Biallelic CEBPA mutations were identified, and a corresponding column was added to the table. Capillary based, quantitative FLT3-ITD testing results were extracted from the corresponding free text reports and added to the table as well.

Chemotherapy orders from the hospital were also available in tabular form and contained information on dosage, medication route, duration, along with therapeutic categories. Demographic data contained information on patient's ages and survival time.

Uses of Disclosed Approach

Below highlights three example use cases illustrating the power of this approach: (1) assessing responses of patients with IDH and/or DNMT3A mutations to therapy with or without an IDH inhibitor, (2) exploring the overlap of the Mixed Phenotype Acute Leukemia (MPAL) and AML with myelodysplasia related changes (AML-MRC) on the basis of flow cytometry and molecular data, and (3) reviewing responses of Acute Myeloid Leukemia (AML) patients to novel venetoclax combination regimens in advance of formal trial results.

IDH/DNMT3A Case Study

IDH1/2 and DNMT3A mutations in AML are associated with opposing epigenetic effects. DNMT3A mutations AML are associated a defect in de-novo DNA methylation resulting in broad hypomethylation. In contrast, IDH1/2 mutations result in a defect in DNA methylation removal and are associated with hypermethylation. A distinct epigenetic signature has been identified in cases with mutations in both IDH and DNMT3A suggestive of an epigenetic antagonism between the forces of hyper and hypomethylation. These AMLs demonstrated RAS pathway perturbation without RAS mutations and an associated increased sensitivity to MEK inhibition in vitro compared with IDH or DNMT3A mutations alone or cases with other mutations.

Given the unique susceptibility of these leukemias, it is useful to understand the clinical courses of these patients compared to those with IDH or DNMT3A mutations alone. The response to IDH inhibitor therapy was of particular interest given the role of RAS mutations as a mechanism of resistance. Clinically, IDH inhibitors became available as standard of care following FDA approval in 2017. It is thus useful to understand the clinical impact of these mutations, given the long use of IDH inhibitors as investigational therapy both in the upfront and relapsed/refractory setting.

An example embodiment queried institutional databases for all patients with either an IDH or DNMT3A mutation at any timepoint. After these data were consolidated the example embodiment was able to model the influence of IDH, DNMT3A, and combined IDH/DNMT3A mutations on overall survival and adjust for the effects of different chemotherapeutic regimens and ELN risk categories.

MPAL and AML-MRC Overlap Case Study

Mixed Phenotype Acute Leukemia (MPAL) presents a unique diagnostic and clinical dilemma. Leukemia is split into cases with a myeloid or a lymphoid lineage, but in 2-5% of cases, features of both lineages are seen simultaneously. MPAL is diagnosed by a strict set of criteria applied to flow cytometry-based immunophenotyping. The diagnostic process is technically complex and requires that other diagnoses are excluded. As a result, there is considerable variability in which cases are diagnosed as MPALs and substantial immunophenotypic overlap with other diagnoses such as AML-MRC or therapy-related AML.

It is thus useful to understand the influence of lineage features on clinical outcomes regardless of the formal clinical diagnosis. Using the disclosed pipeline, an example embodiment extracted features in flow cytometry reports consistent with specific or multiple lineages. Initial diagnostic reports in MPAL and related cases are often ambiguous, so multiple reports were incorporated to determine the final diagnosis. The final dataset included patients with MPAL, AML-MRC, and therapy-related AML, and lineages included myeloid, B/myeloid, T/myeloid, and B/T/myeloid. This information was combined with molecular, cytogenetic, and treatment regimens to perform survival modeling.

To determine the biological variability in immunophenotype independently from the formal diagnosis, the status of specific surface markers was extracted from the flow cytometry report and tabulated. This was achieved by building a custom finite state machine to determine the context in which each marker appeared including positive, negative, bright, and dim. Manual review was used to debug the algorithm. Once the data was extracted, an unsupervised analysis was performed. Supervised analyses including k-means clustering were performed to compare the immunophenotypic features to other markers including clinical diagnosis, cytogenetics, and gene mutations.

HMA/Venetoclax Case Study

For older adult patients with AML, therapeutic options may be limited due to decreased functional status and decreased ability to tolerate intensive chemotherapy. In 2016, combination therapies that included venetoclax, an oral BCL-2 inhibitor, began to be used in this population as an effective alternative to induction therapy. Complete remission rates for older adults treated with venetoclax in combination with the hypomethylating agents (HMAs) decitabine and azacitidine were 60-70% in early phase studies, and favorable responses were also seen for venetoclax given in combination with low-dose cytarabine. This was substantially higher than the standard of care for low intensity therapy.

In November 2018, the FDA granted approval for the use of venetoclax combination therapy in treatment-naïve older adults, but many providers were already using these regimens in patients who had relapsed/refractory disease. Little evidence existed about clinical, molecular, and cytogenetic predictors of response in these patients, and an example embodiment of the disclosed approach was used to assess the utility and effectiveness of these novel combination therapies in a real-world cohort of 86 older adult patients with relapsed/refractory AML.

Using the disclosed system, the example embodiment was able to automate the extraction of data on patients and their molecular status at the time of diagnosis. Variables of interest included patient sample accession number, procedure date, type of next-generation sequencing assay used, variant classes, variant genes, VAFs, chromosomal locations, cDNA changes, start positions, alternative and reference alleles, and date of consent to various IRB research protocols, all of which were associated with patient MRN and name.

Furthermore, the example embodiment was to create structured data from free-text karyotype and FISH reports. This system can automate capture of cytogenetic data including complex/monosomal karyotype, MLL rearrangements, -7/7q, -5/5q, EVI1 rearrangements, t(3;3), inv(3), t(6;9), del(17)/del(17p), and others. Manual curation to confirm the leukNLP output was performed in collaboration with the MSK Cytogenetics Laboratory, but this was streamlined by the availability of the structured reports.

In the example embodiment, an instance of the ComplexHeatmap function was adapted to the disclosed pipeline to create an oncoprint of clinical, molecular, and cytogenetic data, stratified by patient response. This analysis provided clear evidence of the molecular and clinical factors likely to be associated with a response. Cox Proportional Hazards analysis was used to evaluate molecular and cytogenetic predictors of response.

To understand what features might be suggestive of clonal evolution in patients who had had an initial response to venetoclax combination therapy, an embodiment of the disclosed system was used to develop a robust visualization tool to demonstrate relapse kinetics. Moving forward, these data will inform the development of clinical trials that leverage these molecular and cytogenetic predictors of response and relapse to offer more targeted therapies to these patients.

TABLE 2
Tabulations of physician assessed study with cohort of 88 patients
Cytogenetic Categories

| | Parser | | | | | |
|---|---|---|---|---|---|---|
| | | CBF | Complex | Monosomal | Normal | OND |
| Physician | CBF | 6 | 1 | 0 | 0 | 0 |
| | Complex | 1 | 3 | 0 | 0 | 0 |
| | Monosomal | 0 | 0 | 10 | 0 | 0 |
| | Normal | 0 | 0 | 0 | 42 | 0 |
| | OND | 0 | 1 | 1 | 4 | 19 |

| | Sensitivity (TP/TP + FN) | Specificity (TN/TN + FP) |
|---|---|---|
| CBF | 0.85714286 | 0.98765432 |
| Complex | 0.75 | 0.96428571 |
| Monosomal | 1 | 0.98717949 |
| Normal | 1 | 0.91304348 |
| OND | 0.76 | 1 |

AML risk:

| | Parser | | | |
|---|---|---|---|---|
| | | Good | Intermediate | Poor |
| Physician | Good | 17 | 0 | 1 |
| | Intermediate | 3 | 30 | 1 |
| | Poor | 0 | 0 | 35 |
| | Unknown | 0 | 0 | 1 |

| | Sensitivity | Specificity |
|---|---|---|
| | (TP/TP + FN) | (TN/TN + FP) |
| Good | 0.94444444 | 0.95714286 |
| Intermediate | 0.88235294 | 1 |
| Poor | 1 | 0.94339623 |

Data, Processing, and Analysis

Various details of the processing and analysis steps will now be discussed in more detail.

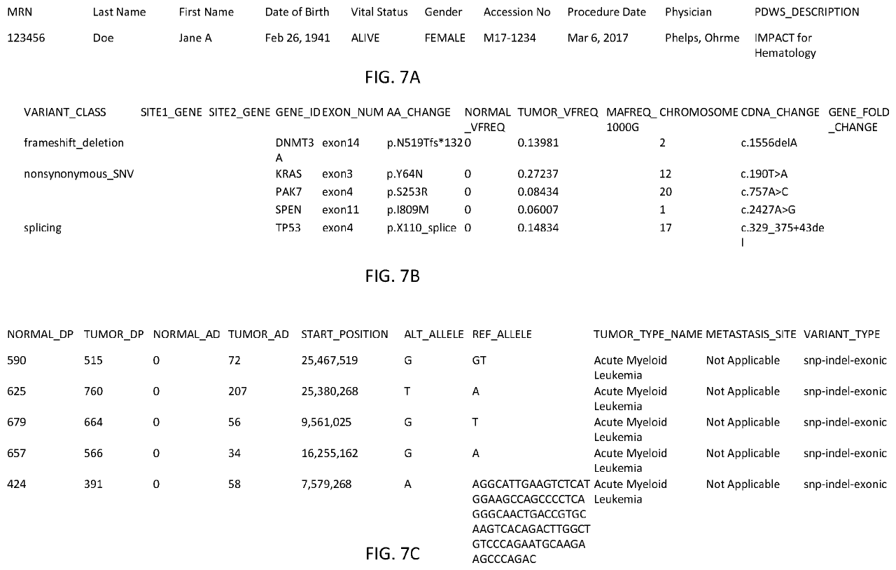

An example DMP report for a next generation sequencing (NGS) result is provided in FIGS. 7A-7C. These are typically structured as a spreadsheet although earlier reports can be converted from raw text to spreadsheet format by a function. This type of report is processed as discussed above—converted from long to wide format (each column is a gene, each row a patient), and then processed to identify CEBPA double mutations.

An example DMP for a FLT3 test is provided below. The underlined portions are extracted into a table that includes columns for FLT3-ITD status (Positive/Negative), FLT3-TKD status (Positive/Negative), FLT3-ITD percentage relative to normal (quantitative), and the FLT3-ITD length.

### PathDoc Version 1.1

MRN: 12345678
Account: 92-03650355
Physician ID: 012345
Physician: Phelps, Ohrme
Accession #: M17-1234
Date of Collection/Procedure/Outside Report: 2/21/2017
Date of Receipt: 2/21/2017
Date of Report: 2/24/2017

### Clinical Diagnosis and History:

AML

### Specimens Submitted:

1: BONE MARROW aliquot from M17-1233

### Diagnostic Interpretation:

POSITIVE for FLT3 Internal Tandem Duplication (ITD)
NEGATIVE for FLT3 TKD mutation
Note: The ITD is 66 bp long. The proportion of FLT3 alleles with the ITD is approximately 15% based on quantitative comparison of the peaks. This value is provided as reference and should be considered approximate as it may also be partly influenced by differences in amplification efficiency of PCR products of different lengths.
A patient without a detectable FLT3 ITD mutation generally has a more favorable prognosis than patients with a FLT3 ITD mutation. Accurate prognosis of a patient with this mutation must be determined together with all other clinical, molecular, and cytogenetic markers.

### Test and Methodology:

Fragment analysis assay for detection of FLT3 ITD (exon 14) and D835 (exon 20) tyrosine kinase domain (TKD) mutations: FLT3 mutations are detected by amplification of exons 14 and 20 of FLT3 by polymerase chain reaction (PCR) in the presence of fluorescently-labeled primers. The TKD PCR product is cut with the EcoRV restriction enzyme. The PCR products are analyzed by capillary electrophoresis on an ABI 3730 DNA Analyzer.
Diagnostic sensitivity: This finding does not exclude the possibility of other FLT3 mutations elsewhere in the gene
Technical sensitivity: This assay cannot detect mutations if the proportion of positive tumor cells in the sample studied is less than 5%. This assay may not detect ITD that are beyond 400 bp in size.

### Lab Notes:

This result cannot be used as sole evidence for or against cancer and has to be interpreted in the context of all available clinical and pathological information.
This test was developed, and its performance characteristics determined, by the Laboratory of Diagnostic Molecular Pathology. It has not been cleared or approved by the U.S. Food and Drug Administration (FDA). The FDA has determined that such clearance is not necessary. This test is used for clinical purposes. Pursuant to the requirements of CLIA '88, our laboratory has established the accuracy and precision of this test.
Additional notes:
DNA quality: Good
Run number: FLT3.123

I ATTEST THAT THE ABOVE DIAGNOSIS IS BASED UPON MY PERSONAL EXAMINATION OF THE SLIDES (AND/OR OTHER MATERIAL), AND THAT I HAVE REVIEWED AND APPROVED THIS REPORT.

Theodore A. Basidium, M.D./CMC

*** Report Electronically Signed Out *** 13:04

An example hematopathology report follows:

### PathDoc Version 1.1

MRN: 00123456
Account: 00-01234567
Physician ID: 012345
Physician: Phelps, Ohrme
Accession #: H16-1234
Date of Collection/Procedure/Outside Report: 12/22/2016
Date of Receipt: 12/22/2016
Date of Report: 12/23/2016

### Clinical Diagnosis & History:

AML. Screen for protocol 12-345.

### Specimens Submitted:

1: RPIC

### Diagnosis:

1. Bone marrow, right posterior iliac crest; biopsy, aspirate, and peripheral blood smear:
   Acute myeloid leukemia, 27% blasts, see comment
COMMENT: Given the prior history of high grade myeloid neoplasm, consistent with refractory anemia with excess blasts-2 (see H16-1233), the findings in this current sample are most consistent with acute myeloid leukemia with myelodysplasia-related changes.

### Bone Marrow Biopsy

Quality: Suboptimal, subcortical small biopsy
Cellularity: 10% on the biopsy in which is not favored to represent the actual cellularity, see aspirate smear
M:E ratio: Slightly decreased
Blasts: difficult to assess due to quality of the material
Myeloid lineage: Left shift of maturation
Erythroid lineage: Exhibit full maturation
Megakaryocytes: Present
Lymphocytes: Scattered
Plasma cells: Scattered

### Bone Marrow Aspirate Smear

Quality: Adequate for evaluation
Differential:
Blasts 27%
Promyelocytes 4%
Myelocytes 15%
Metamyelocytes 5%
Neutrophils/Bands 8%
Monocytes 1%
Eosinophils 1%
Erythroid Precursors 25%
Lymphocytes 14%
Diff: Number of Cells Counted 400
M:E Ratio 1.4
Morphology: Cellularity is best estimated on aspirate smears, approximately 60%. Spicular, cellular aspirate smears show increased number of blasts (medium to large size with round to indented nuclei, fine reticular chromatin, prominent nucleoli, and scant to moderate amount of cytoplasm). Erythroid precursors are increased and dysplastic (nuclear budding, binucleation, nuclear irregularity, nuclear-cytoplasmic asynchrony). Megakaryocytes show occasional dysplastic forms (hypolobation, small size). Histochemical stains: An iron stain is increased for storage iron. No ring sideroblasts seen.

### Peripheral Blood

CBC (11/23/2015):
WBC 1.4 L [4.0-11.0 K/mcL]
RBC 2.52 L [4.20-5.60 M/mcL]
HGB 8.9 L [13.0-17.0 g/dL]
HCT 25.6 L [38.0-52.0%]
MCV 102 H [82-98 fL]
MCH 35.3 H [27.0-33.0 pg]
MCHC 34.8 [31.0-36.5 g/dL]
RDW 19.9 H [11.5-14.5%]
Platelets 10 LL [160-400 K/mcL]
Neutrophil 17.1 L [38.0-80.0%]
Lymph 79.4 H [12.0-48.0%]
Mono 2.8 [0.0-12.0%]
Eos 0.7 [0.0-7.0%]
Baso 0.0 [0.0-1.5%]
Abs Neut 0.2 L [1.5-8.8 K/mcL]
Abs Lymph 1.1 [0.5-5.3 K/mcL]
Abs Mono 0.0 [0.0-1.3 K/mcL]
Abs Eos 0.0 [0.0-0.8 K/mcL]
Abs Baso 0.0 [0.0-0.2 K/mcL]

### Morphology:

RBC: Marked macrocytic anemia with mild anisopoikilocytosis.
WBC: Markedly decreased in number. Rare blasts are seen on scanning.
Platelet: Markedly decreased in number.

### Immunohistochemistry

Rare blasts are positive for CD34 and CD117.

### Flow Cytometric Analysis, Bone Marrow (F16-1234)

Abnormal myeloid blast population detected.
Flow cytometry identifies an abnormal blast population with an immunophenotype similar to that seen in prior sample (F16-1233) have abnormal expression of CD13 (uniform), CD33 (bright), CD34 (absent), HLA-DR (uniform), CD117 (partial dim), CD123 (uniform), with normal expression of CD4, CD38, CD45 and CD71 without CD2, CD5, CD7, CD11b, CD14, CD15, CD16, CD19, CD56 or CD64. The abnormal myeloid blasts represent 27.4% of WBC. In addition, CD14 absent immature monocytes are slightly expanded, representing 7.7% of totally WBC. The overall blast count is estimated at 35.1% of WBC. The findings are diagnostic for persistent AML.

### Cytogenetic Studies

Cytogenetic analysis will be reported separately. See separate report,
CG16-1234.

### Molecular Studies

Molecular analysis will be reported separately. See separate report,
M16-12345/M16-12346.

The interpretation of these results is based in part on the decalcification procedure performed.

I ATTEST THAT THE ABOVE DIAGNOSIS IS BASED UPON MY PERSONAL EXAMINATION OF THE SLIDES (AND/OR OTHER MATERIAL), AND THAT I HAVE REVIEWED AND APPROVED THIS REPORT.

Theodore Basidium, MD, PhD/WXS

*** Report Electronically Signed Out *** 10:45

### Gross Description:

Arnold Petri, H.T.
1) The specimen is received in formalin, labeled ““RPIC””, and consists of one piece(s) of red-brown, bony tissue measuring 0.7 cm in length. The specimen is decalcified and entirely submitted.

### Summary of Sections:

BM—bone marrow

### Summary of Sections:

Part 1: RPIC

| Blocks | Block Designation | PCs |
|---|---|---|
| 1 | BM | 1 |

### Special Studies:

| Result | Special Stain | Comment |
|---|---|---|
| Unst_Norm | | |
| CD34 | | |
| CD117 | | |
| Unst_Norm | | |
| Unst_Norm | | |
| Unst_Norm | | |

All controls are satisfactory. Some of the immunohistochemistry and Insitu Hybridization tests were developed and their performance characteristics were determined by the Department of Pathology. They have not been cleared or approved by the US Food and Drug Administration. The FDA has determined that such clearance or approval is not necessary.

These tests are used for clinical purposes. They should not be regarded as investigational or for research. This laboratory is certified under the Clinical Laboratory Improvement Amendments of 1988 (CLIA '88) as qualified to perform high complexity clinical laboratory testing.

For FDA Approved/Cleared Antibodies Only:

All controls are satisfactory. Ventana's PATHWAY anti-HER-2/neu is an FDA-approved rabbit monoclonal primary antibody (clone 4B5) directed against the internal domain of the c-erbB-2 oncoprotein (HER2) for immunohistochemical detection of HER2 protein overexpression in breast cancer tissue routinely processed for histologic evaluation. Results are reported in accordance with the ASCO/CAP guideline recommendations for HER2 testing in breast cancer (J Clin Oncol. 2013 Nov. 1; 31(31):3997-4013). ER and PR are monoclonal antibodies which are FDA-cleared. and cytogenetics report.

An example cytogenetic report is provided here. It is noted that the cytogenetics report includes a FISH section. SNP arrays are used infrequently but would appear below the FISH section. This may be processed using the expression patterns disclosed herein.

### PathDoc Version 1.1

MRN: 00012345
Account: 12345678
Physician ID: 012345
Physician: Phelps, Ohrme
Accession #: CG18-1234
Date of Collection/Procedure/Outside Report: 9/16/2018
Date of Receipt: 9/17/2018
Date of Report: 9/30/2018

### Clinical Diagnosis & History:

THERAPY-RELATED MDS with t(3;3)/EVI1 REARRANGEMENT

### Specimens Submitted:

1: BONE MARROW

### Preparation:

### Karyotype Analysis

Preparation information: 24 HRS
Sample Preparation: ADEQUATE (APPROXIMATELY 300-400 BANDS; G-BANDING)

### Fish Analysis

Preparation information: 24 HRS
Sample Preparation: POOR
Number of cells analyzed: 100-300
Probe used (Vendor), chromosome localization of target gene, cut-off for normal variation in BM/PB:
EVI1 Tricolor Breakapart (Leica Biosystems), EVI1 (3q26.2), 1.4% for rearrangement
D7S486/CEP 7 (Abbott Molecular), D7S486 (7q31), 1.4% for D7S486 deletion and 3% for loss of chromosome 7
LSI ETV6 Dual Color Breakapart (Abbott Molecular), ETV6 (12p13.2), 1.4% for rearrangement, 2% for deletion

### Test Performed:

CHROMOSOME AND FISH ANALYSIS.

### Test Results:

### Karyotype Analysis

Number of cells counted: 20
Number of cells analyzed: 20
Number of cells karyotyped: 3
Modal chromosome number(s): 46
Modal karyotype(s): 46,XY,t(3;3)(q21;q26.2),del(7)(q22q34)[20]

### Fish Analysis

Interphase/Nuclear In Situ Hybridization [ISCN 2016]: nuc
ish(D3S1243,TERC,D3S1564)x2(D3S1243 sep TERC con
D3 S1564x1)[80/100],(D7Z 1 x2,D7S486x1)[260/300],(ETV6x2)[300]

### Diagnostic Interpretation:

### Karyotype Analysis:

Chromosome analysis detected the previously observed t(3;3) and deletion of 7q in all twenty metaphases. This finding is consistent with this patient's persistent therapy-related myeloid neoplasia (H20-5150).
Previous study: 7/13/2020, CG20-3198.

### Fish Analysis:

Rearrangement of EVIL (3q26.2) detected in 80% of cells.
Deletion of 7q31 detected in 86.7% of cells.
No evidence of ETV6 (12p13.2) deletion.
A correlation with other studies is recommended for a precise diagnosis.

### Note:

### Disclaimer:

Karyotype analysis may not detect subtle translocations, deletions, inversions or other chromosomal abnormalities that are beyond the resolution limits of the banding technology used. This assay is not a ““““stand-alone”””” test for the diagnosis of cancer, on the other hand, a normal karyotype does not rule out cancer.

The FISH test was developed and its performance determined by the Laboratory of Cytogenetics. Although it has not been cleared or approved by the U.S. Food and Drug administration, the FDA has determined that such clearance or approval is not necessary. Pursuant to the requirements of CLIA '88, however, this laboratory has established and verified the test's accuracy and precision; therefore this test is used for clinical purposes.

Theodore Basidium, PhD, FACMG

Clinical Cytogeneticist

Clinical Molecular Geneticist

I ATTEST THAT THE ABOVE IMPRESSION IS BASED UPON MY PERSONAL EXAMINATION OF THE KARYOTYPE AND/OR FISH IMAGE(S), AND THAT I HAVE REVIEWED AND APPROVED THIS REPORT.

Arnold Petri, M.D./JMH

*** Report Electronically Signed Out *** 21:34″

With respect to the example cytogenetics report, modal karyotype may be processed by a karyotype parser. Karyotype diagnostic analysis may be optionally processed using expression patterns (see below). FISH diagnostic analysis may be processed using below expression patterns (below) and integrated with the karyotype data.

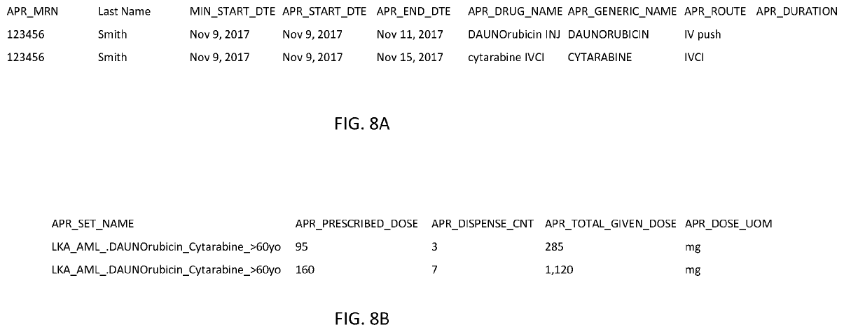

An example of a chemotherapy structured (raw) data is provided in FIGS. 8A and 8B (each figure shows all rows, but columns stretch across FIGS. 8A and 8B). Regimens are provided in FIGS. 9A-9D (in which each figure includes all columns, but rows are split up across figures). Using the regimens of FIGS. 9A-9D, the data in FIGS. 8A and 8B would be converted to ‘7+3’ using the row 52:

| drugs | regimen | type | target | investigational |
|---|---|---|---|---|
| cytarabine, | 7 + 3 | induction | none | FALSE |
| daunorubicin | | | | |

Survival time corresponds to how long a patient is alive following the diagnosis. It is calculated by subtracting the date of death (or censoring) from the date of diagnosis.

For AML cytogenetic categories, the National Comprehensive Cancer Network (NCCN) guidelines for AML classification may be used (see Table 1 above). Entries in Table 1 without a gene name is a cytogenetic category. In the good risk box, t(8;21) and inv(16) are often referred to as ‘Core binding factor’ and t(15;17) is ‘APL’ or acute promyelocytic leukemia. Referring to FIG. 5, the double solid lined boxes (the boxes starting with the text “t(15;17),” “Normal,” and “Monosomal”) are cytogenetic categories.

Using ELN criteria on the cytogenetics example above:
46,X,Y,t(3;3)(q21;q26.2) [14]/idem,del7(q22q34)[4], idem, add(17)(p12)[2]

This includes inv(3) or t(3;3) which is in the poor risk group. So it would be poor risk by ELN guidelines as described in Table 1.

### Expression Patterns

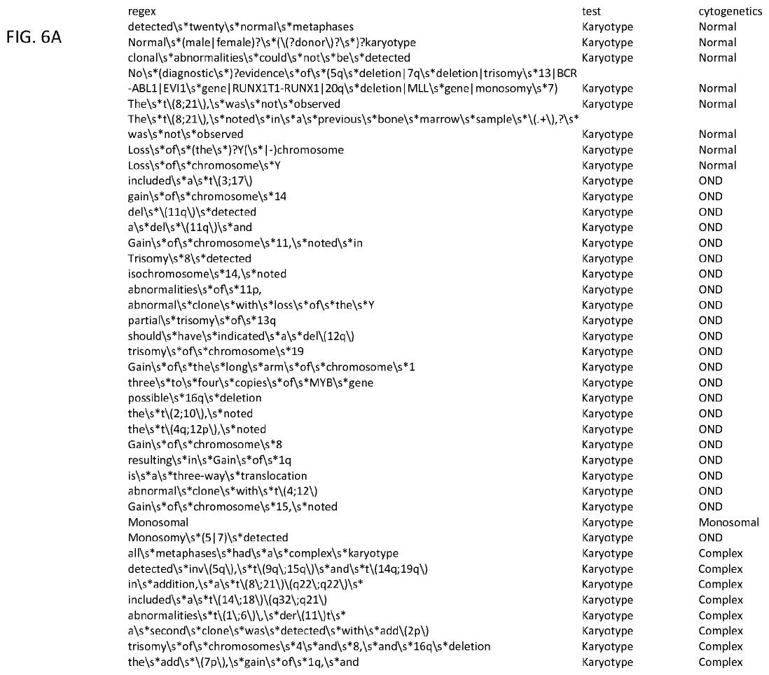

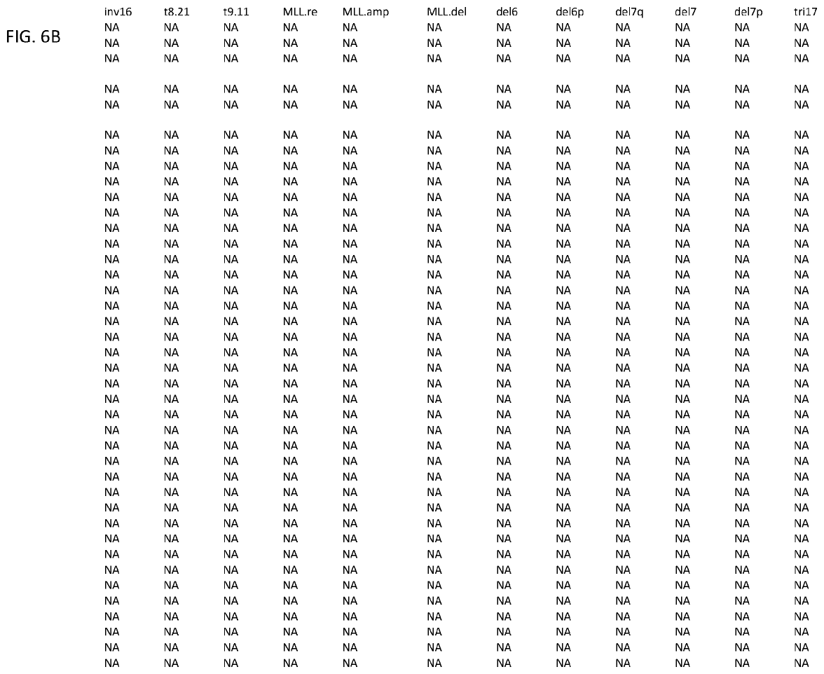

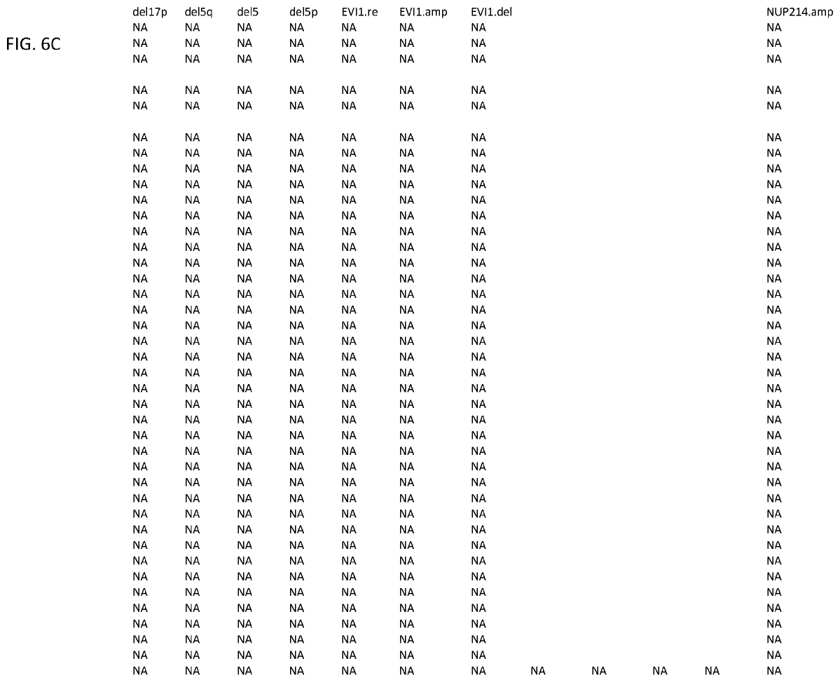

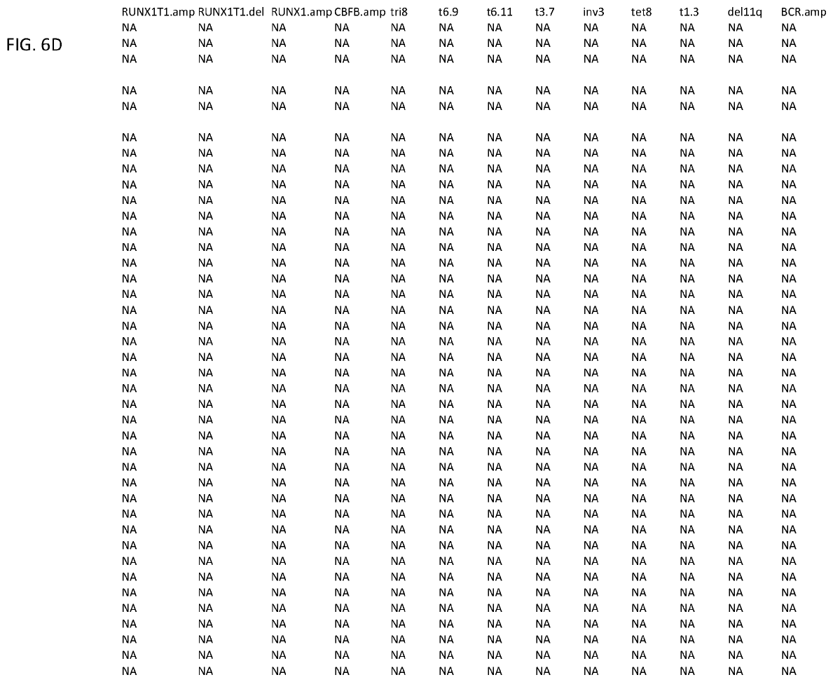

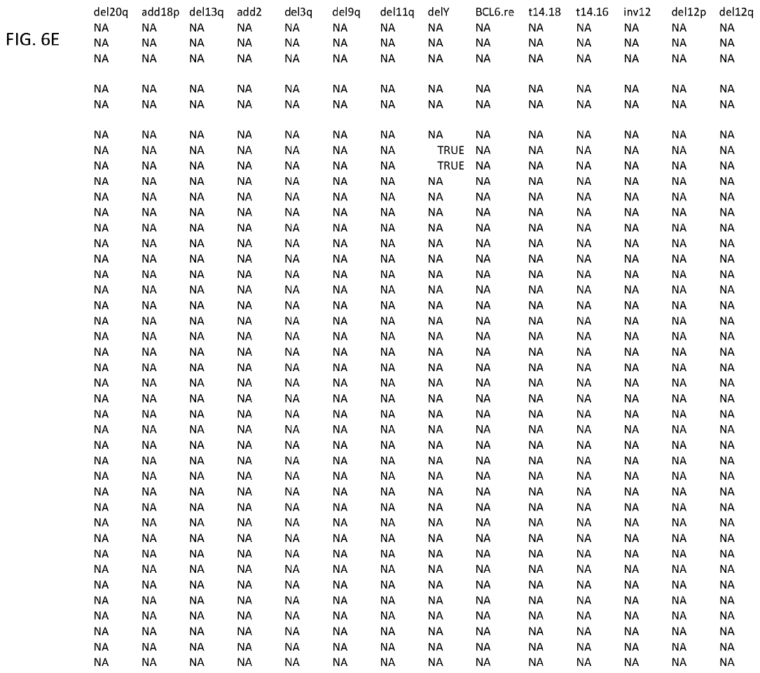

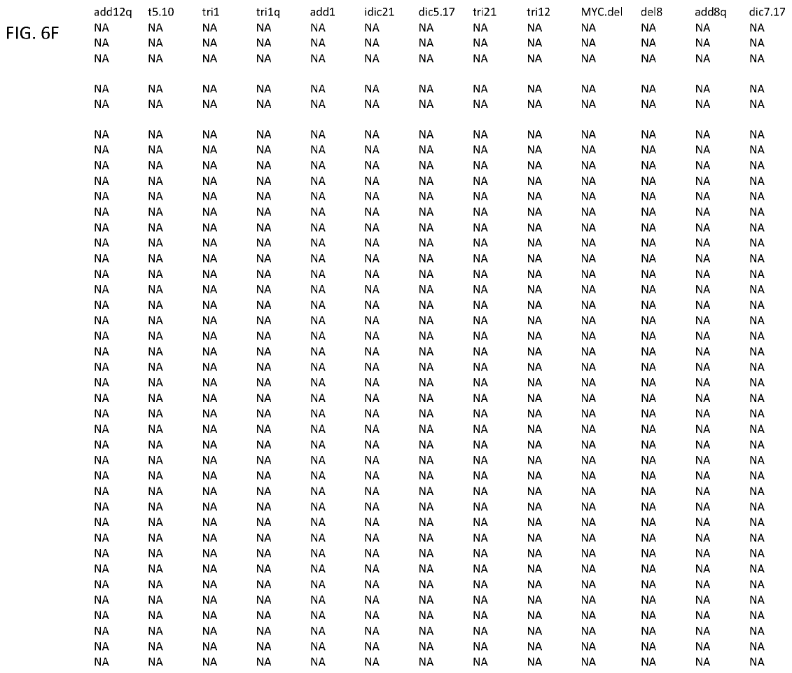

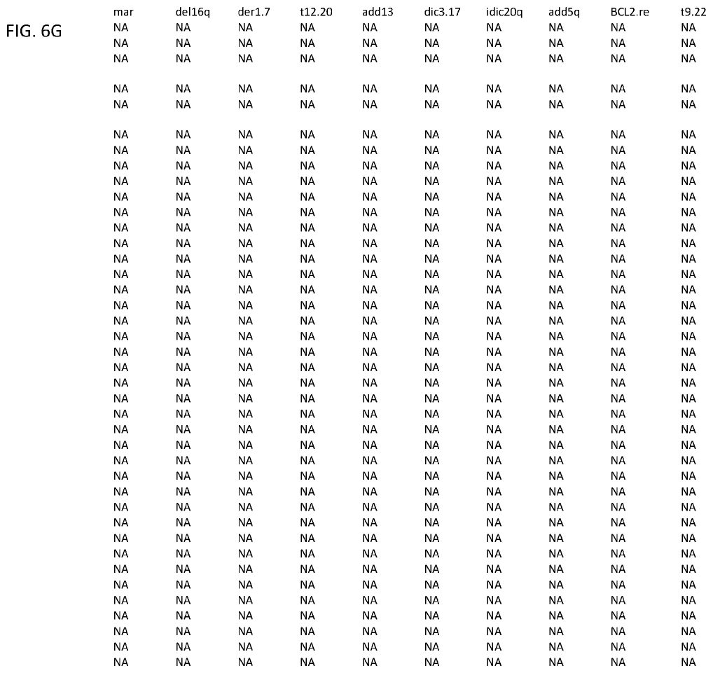

Example expression patterns (used interchangeably with conditional patterns) for reports is provided below. In this example, if the pattern in column 1 (‘regex’) is found in the test described in column 2 (‘test’), then the subsequent columns are assigned in the table corresponding to that patient record. For example in row 1 if ‘detected\s*twenty\s*normal\s*metaphases’ matches the ‘Karyotype’ record (i.e., if the expression patter in row 1 is “triggered”), cytogenetics are assigned as ‘Normal’ and all other features are assigned an ‘NA’ (i.e., not defined). More than one pattern may match a given record, and in that case the features are assigned according to an inputted priority vector. For example ‘CBF’ in cytogenetics is priority 1 (never replaced) while ‘Normal’ is replaced by anything else if that anything else is also found (last priority). In the True/False columns, True supersedes False, and anything supersedes NA. With respect to example pattern above from row 1, the term “detected” would be followed by the term “twenty” in the same sentence and separated by any or no white space (as indicated by the operator \s*). This pattern would not match in the following sentence from the example cytogenetic report provided herein because of a lack of the term “normal”: “Chromosome analysis detected the previously observed t(3;3) and deletion of 7q in all twenty metaphases. This finding is consistent with this patient's persistent therapy-related myeloid neoplasia (H20-5150).” Ten example expression patterns are provided in Table 3 below. FIGS. 6A-6G provide 40 example rows in a table, with all 40 rows included in each figure, and the columns extended across the figures.

TABLE 3
Example Expression Patterns

Regex
detected\s*twenty\s*normal\s*metaphases
Normal\s*(male|female)?\s*(\(?donor\)?\s*)?karyotype
clonal\s*abnormalities\s*could\s*not\s*be\s*detected
No\s*(diagnostic\s*)?evidence\s*of\s*(5q\s*deletion|7q\s*deletion|trisomy\s*13|BCR-
ABL1|EVI1\s*gene|RUNX1T1-RUNX1|20q\s*deletion|MLL\s*gene|monosomy\s*7)
The\s*t\(8;21\),\s*was\s*not\s*observed
The\s*t\(8;21\),\s*noted\s*in\s*a\s*previous\s*bone\s*marrow\s*sample\s*\(.+\),?\s*was\s
*not\s*observed
Loss\s*of\s*(the\s*)?Y(\s*|−)chromosome
Loss\s*of\s*chromosome\s*Y
included\s*a\s*t\(3;17\)
gain\s*of\s*chromosome\s*14

With respect to Table 3, the expression patterns use the following operators: “\s” indicating white space (space, tab, new line, etc.); “\” indicating the subsequent character is to be taken as literal (except in the case of a special pattern such as ‘\s’); “1” indicating or; “*” indicating any 0 or more instances of the previous pattern are matched; and “?” indicating 0 or 1 of the previous pattern are matched.

Example Code

library(data.table)
library(leukNLP)
# directories
dir.root = ′/Volumes/HOME/lab/nlp_project′
dir.scratch = file.path(dir.root, ′scratch′)
dir.hipaa = file.path(dir.root, ′hipaa′)
dir.labels = file.path(dir.root, ′hipaa/labels′)
## load pathology data
dt.path.orig = loadPathology.dt(file.path(dir.hipaa, ′pathology.txt′))
vec.date.cols = c(′Date.of.Birth′, ′Pathology.Procedure.Date′, ′Procedure. Date′)
# load DMP data
vec.date.cols=c(′Procedure.Date′)
dt.path.variants = loadVariants.dt(file.path(dir.tables.main, dmp.txt′), vec.date.cols)
# process FLT3 data
dt.flt3 = processFlt3.dt(dt.path.orig, col.date=’Pathology.Procedure.Date’,
col.report=’Pathology.Report.Text’, col.specimen=’specimen’)
# define possible report labels within each report category
lst.reportType = list( )
lst.reportType[[′hemePath′] = c(′Hematopathology Report′, ′Pathology-Bone Marrow′,
′Surgical Pathology′)
lst.reportType[′hemeConsult′]] = c(′Hematopathology Departmental Consult′,
′Hematopathology Consult′)
lst.reportType[[′cytogenetics′]] = c(′Cytogenetics′)
lst.reportType[′dmp′]] = c(′Diagnostic Molecular Pathology′)
lst.reportType[[′flow′]] = c(′Flow Cytometry Report′)
# get subset of hematopathology reports using the label list above
dt.path.heme = dt.path.orig[Pathology.Report.Type %in% lst.reportType[[′hemePath′]], ]
dt.path.heme[, diagnosis:=sapply(Pathology.Report.Text, extractDiagnosis.str)]
dt.path.heme[, HGMN:=sapply(diagnosis,
leukNLP::id.high_grade_myeloid_neoplasm.bool)]
dt.path.heme[, AML:=sapply(diagnosis, id.acute_myeloid_leukemia.bool)]
dt.path.heme[, MDS:=sapply(diagnosis, id.myelodysplastic_syndrome.bool)]
dt.path.heme[, MPN:=sapply(diagnosis, id.myeloproliferative_neoplasm.bool)]
# get the subset of outside consults; extract and process outside procedure dates
dt.path.consult = processConsults.dt(dt.path.orig[Pathology.Report.Type %in%
lst.reportType[[′hemeConsult′]],])
dt.path.consult[, diagnosis:=sapply(Pathology.Report.Text, extractDiagnosis.str)]
dt.path.consult[, HGMN:=sapply(diagnosis,
leukNLP::id.high_grade_myeloid_neoplasm.bool)]
dt.path.consult[, AML:=sapply(diagnosis, id.acute_myeloid_leukemia.bool)]
dt.path.consult[, MDS:=sapply(diagnosis, id.myelodysplastic_syndrome.bool)]
dt.path.consult[, MPN:=sapply(diagnosis, id.myeloproliferative_neoplasm.bool)]
## Load demographics
dt.demographics = loadTable.dt(file.path(dir.tables.main, ′Demographics.txt′), vec.date.cols)
## Get dates of diagnosis by getting the first path report regardless of diagnosis
vec.diagnoses = c(′HGMN′, ′AML′, ′MDS′, ′MPN′)
dt.dates.heme.dx =
processPathDemo.dt(dt.path.heme[AML==T|HGMN==T|MDS==T|MPN==T,],
dt.demographics, col.date=′Pathology.Procedure.Date′, vec.otherCols=vec.diagnoses)
dt.dates.consults.dx =
processPathDemo.dt(dt.path.consult[AML==T|HGMN==T|MDS==T|MPN==T,],
dt.demographics, col.date=′procedure_date′, vec.otherCols=vec.diagnoses)
dt.dates.dx.orig = rbind(dt.dates.heme.dx, dt.dates.consults.dx)
# Get date of diagnosis by taking the first one of either in house or consult
dt.dates.dx = dt.dates.dx.orig[, .SD[which.min(dx.date)], by=′MRN′]
# MRN vector for these patients
vec.mrns.AML = dt.dates.dx[AML==T, unique(MRN)]
## Extract cytogenetic data
lbl.cyto = fread(system.file(′extdata′, ′regex_cytogenetics.txt′, package=′leukNLP′))
# leave out CBF subtypes and other columns not informing risk
vec.cols.cyto = setdiff(colnames(lbl.cyto), c(′regex′, ′test′, ′t8.21′, ′inv16′))
vec.cyto.priority = c(′CBF′=1, ′Monosomal′=2, ′Complex′=3, ′OND′=4, ′Normal′=5)
# Add columns with Karyotype, FISH, and SNP data extracted
dt.path.cyto.orig = processCyto.dt(dt.path.orig[Pathology.Report.Type==′Cytogenetics′,])
# identify relevant cytogenetic abnormalities from reports (grepls)
dt.path.cyto = assignCyto.dt(dt.path.cyto.orig[MRN %in% vec.mrns.AML, ], lbl.cyto,
vec.cols.cyto, vec.cyto.priority)
# get diagnostic subsets
dt.path.cyto.dx = getSubset.dx.dt(dt.dates.dx, dt.path.cyto)
# associate ELN risk with each report
lbl.eln.cyto = fread(system.file(′extdata′, ′risk_cyto_ELN.txt′, package=′leukNLP′))
lbl.eln.dmp = fread(system.file(′extdata′, ′risk_dmp_ELN.txt′, package=′leukNLP′))
vec.eln.priority = c(′Good′=1, ′Poor′=2, ′Intermediate′=3)
dt.path.variants.dx = getSubset.dx.dt(dt.dates.dx, dt.path.variants,
col.data.date=′Procedure.Date′)
lst.path.variants.dx.table = variantsToTable.lst(dt.path.variants.dx)
# merge DMP with dedicated FLT3 assay
dt.flt3.merged = merge(lst.path.variants.dx.table$labeled, dt.flt3[, list(MRN,
Procedure.Date=Pathology.Procedure.Date, cap.itd-FLT3.ITD, cap.tkd=FLT3.TKD,
cap.multiple=FLT3.multiple, ITD.length, ITD.pct)], by=c(′MRN′, ′Procedure.Date′))
# update FLT3 status and FLT3.ITD level using dedicated assay data
dt.flt3.merged[, FLT3.ngs:=FLT3]
dt.flt3.merged[, FLT3.ITD.ngs:=FLT3.ITD]
dt.flt3.merged[, FLT3:=(FLT3.ngs==T|cap.itd==′POSITIVE′|cap.tkd==′POSITIVE′)]
dt.flt3.merged[, FLT3.ITD:=ifelse(is.na(ITD.length), ′Absent′, ifelse(ITD.pct>=50, ′High′,
′Low′))]
# merge with cytogenetics
dt.path.cytoDmp.dx= merge(dt.path.cyto.dx, dt.flt3.merged, by=′MRN′, all=T)
# assign risk
dt.path.cytoDmp.dx[, AML.Risk:=assignRisk.AML.str(.SD, lbl.eln.cyto, lbl.eln.dmp,
vec.who.priority, vec.cols.cyto), by=c(′MRN′, ′procedure.date′)]
# plot mutations / cytogenetics as heatmap / oncoprint
library(RColorBrewer)
library(ComplexHeatmap)
vec.cols.dmp = setdiff(colnames(lst.path.variants.dx.table$variants), c(′MRN′,
′Procedure.Date′))
mat.cytoDmp.dx.op = as.matrix(lst.path.variants.dx.table$variants[, .SD,
.SDcols=vec.cols.dmp], rownames=lst.path.variants.dx.table$variants[, MRN])
vec.variantClasses = dt.path.variants.dx[GENE_ID!=″, unique(VARIANT_CLASS)]
names(vec.variantClasses) = vec.variantClasses
vec.colors.op = setNames(brewer.pal(length(vec.variantClasses), ′Set1′), vec.variantClasses)
lst.alter_fun = lapply(vec.variantClasses, function(str.class) {function(x, y, w, h) {grid.rect(x,
y, w-unit(0.2, ″mm″), h-unit(0.2, ″mm″), gp = gpar(fill = vec.colors.op[str.class], col =
NA))}})
lst.alter_fun[[′background′]] = function(x, y, w, h) { grid.rect(x, y, w-unit(0.5, ″mm″), h-
unit(0.5, ″mm″), gp = gpar(fill = ′gray20′, col = NA))}
pdf(file.path(dir.scratch, ′oncoprint.pdf), width=10, height=15, onefile=F)
oncoPrint(t(mat.cytoDmp.dx.op), get_type=function(x) {strsplit(x, ′;′)[[1]]},
alter fun=lst.alter_fun, col=vec.colors.op, column_title=′Oncoprint for NLP project patients’)
dev.off( )
# Flow
lbl.flow = fread(system.file(‘extdata’, ‘regex_flow.txt’, package=’leukNLP’))
vec.cols.flow = setdiff(colnames(lbl.flow), ‘regex’)
dt.path.flow.orig = processFlow.dt(dt.path.orig[Pathology.Report. Type %like% ′Flow′,])
dt.path.flow = assignFlow.dt(dt.path.flow.orig[MRN %in% vec.mrns.AML,], lbl.flow,
vec.cols.flow)
## load treatment data
lbl.intensity = fread(system.file(′extdata′, ′chemo_intensity.txt′, package=′leukNLP′))
setkey(lbl.intensity, ′drug′)
lbl.drugs = fread(system.file(′extdata′, ′chemo_drugs.txt′, package=′leukNLP′))
setkey(lbl.drugs, ′Drug.Name′)
lbl.drugs.admin = fread(system.file(′extdata′, ′chemo_continuous.txt′, package=′leukNLP′))
setkey(lbl.drugs.admin, ′drug′)
# routes we′re interested in
vec.routes = c(′IVPB′, ′IV push′, ′Oral′, ′subcutaneous′, ′oral′, ′IVCI′, ′subcutaneous.′, ′ivbp′)
dt.chemo.routes = loadChemo.routes.dt(′chemoroute.txt′, lbl.drugs, vec.routes)
# load the chemo dispenses table
dt.chemo.dispenses = loadChemo.dispenses.dt(′chemo_dispenses.txt′, lbl.drugs)
# remove unlabeled intrathecal doses by drug / dosage (can be adjusted as needed)
dt.chemo.dispenses = dt.chemo.dispenses.orig[!(drug==′methotrexate′ & Normalized.Dose
%in% 10:15) & !(drug==′cytarabine′ & Normalized.Dose==70),]
# merge route / dispense tables using MRN, date, and dose
dt.chemo.orig = merge(dt.chemo.dispenses[, list(MRN, drug, Dose, Unit,
Start.Date=Order.Start.Date, Stop.Date=Order.Stop.Date)], dt.chemo.routes[, list(MRN, drug,
Dose, Unit, Start.Date, Route)], by=c(′MRN′, ′drug′, ′Dose′, ′Unit′, ′Start.Date′), all.x=T)
# select regimens given after AML diagnosis (0-30 day buffer here)
dt.chemo.dx = merge(dt.chemo.orig[MRN %in% vec.mrns.AML, ], dt.dates.dx, by=′MRN′)
dt.chemo[, days.postDiagnosis:=as.numeric(difftime(Start.Date, dx.date), units=′days′)]
dt.chemo.dx = dt.chemo[days.postDiagnosis %between% c(0, 30), ]
# convert drugs into regimens
lbl.chemo.regimens = fread(system.file(′extdata′, ′chemo_regimens.txt′, package=′leukNLP′))
dt.chemo.regimens = processChemo.regimens.dt(dt.chemo.dx, lbl.chemo.regimens,
lbl.drugs.admin[continuous==T, drug])
dt.chemo.regimens[order(MRN, start), cycle:=1:.N, by=c(′MRN′, ′drugs′)]
### Survival modeling using the data above ###
## load patient data
vec.os.status = c(′ALIVE′=1, ′DECEASED′=2)
dt.survival.orig = loadSurvival.dt(file.path(dir.hipaa, ′survival_data.txt′), vec.os.status)
dt.survival = processSurvival.dt(dt.survival.orig, dt.dates.dx)
## Add treatment type to survival table
dt.survival[, induction:=ifelse(MRN %in% dt.chemo.regimens[type==′induction′,
unique(MRN)], T, F)]
dt.survival[, low.intensity:=ifelse(MRN %in% dt.chemo.regimens[type==′low.intensity′,
unique(MRN)], T, F)]
dt.survival[, investigational:=ifelse(MRN %in% dt.chemo.regimens[investigational==′yes′,
unique(MRN)], T, F)]
# add risk
o.risk = c(′Intermediate′, ′Poor′, ′Good′)
dt.survival = merge(dt.survival, dt.path.cytoDmp.dx, by=′MRN′, all.x=T)
dt.survival[, AML.Risk:=factor(AML.Risk, levels=o.risk)]
## Survival modeling
# AML risk
coxph(Surv(os.months, os.status) ~ AML.Risk, data=dt.survival)
# Induction therapy
survfit(Surv(os.months, os.status) ~ induction, data=dt.survival)
# Low intensity therapy
survfit(Surv(os.months, os.status) ~ low.intensity, data=dt.survival)
# Investigational therapy
survfit(Surv(os.months, os.status) ~ investigational, data=dt.survival)

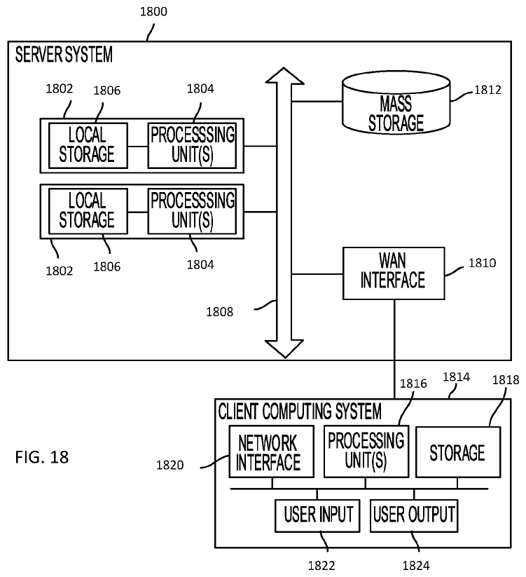

Various operations described herein can be implemented on computer systems having various design features. FIG. 18 shows a simplified block diagram of a representative server system 1800 and client computer system 1814 usable to implement certain embodiments of the present disclosure. In various embodiments, server system 1800 or similar systems can implement services or servers described herein or portions thereof. Client computer system 1814 or similar systems can implement clients described herein.

Server system 1800 can have a modular design that incorporates a number of modules 1802 (e.g., blades in a blade server embodiment); while two modules 1802 are shown, any number can be provided. Each module 1802 can include processing unit(s) 1804 and local storage 1806.

Processing unit(s) 1804 can include a single processor, which can have one or more cores, or multiple processors. In some embodiments, processing unit(s) 1804 can include a general-purpose primary processor as well as one or more special-purpose co-processors such as graphics processors, digital signal processors, or the like. In some embodiments, some or all processing units 1804 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1804 can execute instructions stored in local storage 1806. Any type of processors in any combination can be included in processing unit(s) 1804.

Local storage 1806 can include volatile storage media (e.g., conventional DRAM, SRAM, SDRAM, or the like) and/or non-volatile storage media (e.g., magnetic or optical disk, flash memory, or the like). Storage media incorporated in local storage 1806 can be fixed, removable or upgradeable as desired. Local storage 1806 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1804 need at runtime. The ROM can store static data and instructions that are needed by processing unit(s) 1804. The permanent storage device can be a non-volatile read-and-write memory device that can store instructions and data even when module 1802 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

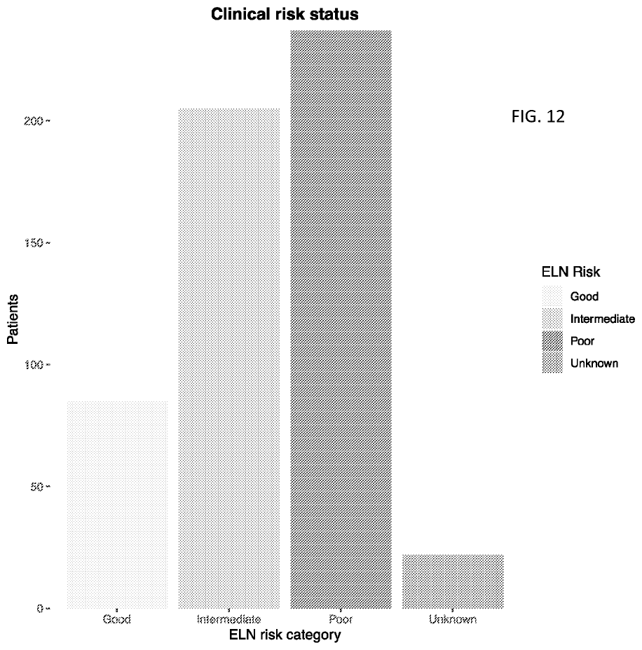

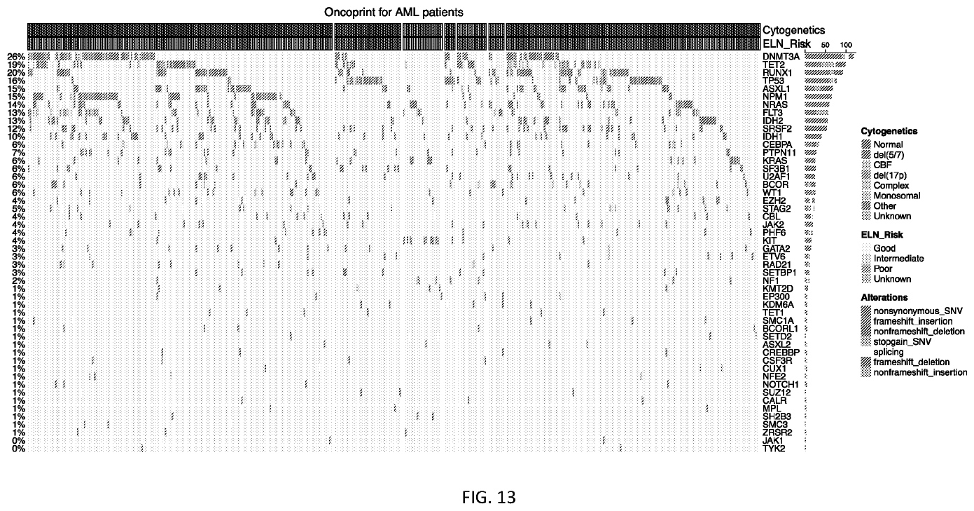

In some embodiments, local storage 1806 can store one or more software programs to be executed by processing unit(s) 1804, such as an operating system and/or programs implementing various server functions or computing functions, such as any functions of any components of FIGS. 1 and 12 or any other computing device, computing system, and/or sensor identified in this disclosure.

“Software” refers generally to sequences of instructions that, when executed by processing unit(s) 1804 cause server system 1800 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1804. Software can be implemented as a single program or a collection of separate programs or program modules that interact as desired. From local storage 1806 (or non-local storage described below), processing unit(s) 1804 can retrieve program instructions to execute and data to process in order to execute various operations described above.

In some server systems 1800, multiple modules 1802 can be interconnected via a bus or other interconnect 1808, forming a local area network that supports communication between modules 1802 and other components of server system 1800. Interconnect 1808 can be implemented using various technologies including server racks, hubs, routers, etc.

A wide area network (WAN) interface 1810 can provide data communication capability between the local area network (interconnect 1808) and a larger network, such as the Internet. Conventional or other activities technologies can be used, including wired (e.g., Ethernet, IEEE 802.3 standards) and/or wireless technologies (e.g., Wi-Fi, IEEE 802.11 standards).

In some embodiments, local storage 1806 is intended to provide working memory for processing unit(s) 1804, providing fast access to programs and/or data to be processed while reducing traffic on interconnect 1808. Storage for larger quantities of data can be provided on the local area network by one or more mass storage subsystems 1812 that can be connected to interconnect 1808. Mass storage subsystem 1812 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1812. In some embodiments, additional data storage resources may be accessible via WAN interface 1810 (potentially with increased latency).

Server system 1800 can operate in response to requests received via WAN interface 1810. For example, one of modules 1802 can implement a supervisory function and assign discrete tasks to other modules 1802 in response to received requests. Conventional work allocation techniques can be used. As requests are processed, results can be returned to the requester via WAN interface 1810. Such operation can generally be automated. Further, in some embodiments, WAN interface 1810 can connect multiple server systems 1800 to each other, providing scalable systems capable of managing high volumes of activity. Conventional or other techniques for managing server systems and server farms (collections of server systems that cooperate) can be used, including dynamic resource allocation and reallocation.

Server system 1800 can interact with various user-owned or user-operated devices via a wide-area network such as the Internet. An example of a user-operated device is shown in FIG. 18 as client computing system 1814. Client computing system 1814 can be implemented, for example, as a consumer device such as a smartphone, other mobile phone, tablet computer, wearable computing device (e.g., smart watch, eyeglasses), desktop computer, laptop computer, and so on.

For example, client computing system 1814 can communicate via WAN interface 1810. Client computing system 1814 can include conventional computer components such as processing unit(s) 1816, storage device 1818, network interface 1820, user input device 1822, and user output device 1824. Client computing system 1814 can be a computing device implemented in a variety of form factors, such as a desktop computer, laptop computer, tablet computer, smartphone, other mobile computing device, wearable computing device, or the like.

Processor 1816 and storage device 1818 can be similar to processing unit(s) 1804 and local storage 1806 described above. Suitable devices can be selected based on the demands to be placed on client computing system 1814; for example, client computing system 1814 can be implemented as a “thin” client with limited processing capability or as a high-powered computing device. Client computing system 1814 can be provisioned with program code executable by processing unit(s) 1816 to enable various interactions with server system 1800 of a message management service such as accessing messages, performing actions on messages, and other interactions described above. Some client computing systems 1814 can also interact with a messaging service independently of the message management service.

Network interface 1820 can provide a connection to a wide area network (e.g., the Internet) to which WAN interface 1810 of server system 1800 is also connected. In various embodiments, network interface 1820 can include a wired interface (e.g., Ethernet) and/or a wireless interface implementing various RF data communication standards such as Wi-Fi, Bluetooth, or cellular data network standards (e.g., 3G, 4G, LTE, 5G, etc.).

User input device 1822 can include any device (or devices) via which a user can provide signals to client computing system 1814; client computing system 1814 can interpret the signals as indicative of particular user requests or information. In various embodiments, user input device 1822 can include any or all of a keyboard, touch pad, touch screen, mouse or other pointing device, scroll wheel, click wheel, dial, button, switch, keypad, microphone, and so on.

User output device 1824 can include any device via which client computing system 1814 can provide information to a user. For example, user output device 1824 can include a display-to-display images generated by or delivered to client computing system 1814. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1824 can be provided in addition to or instead of a display. Examples include indicator lights, speakers, tactile “display” devices, printers, haptic devices (e.g., tactile sensory devices may vibrate at different rates or intensities with varying timing), and so on.

Some embodiments include electronic components, such as microprocessors, storage and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1804 and 1816 can provide various functionality for server system 1800 and client computing system 1814, including any of the functionality described herein as being performed by a server or client, or other functionality associated with message management services.

It will be appreciated that server system 1800 and client computing system 1814 are illustrative and that variations and modifications are possible. Computer systems used in connection with embodiments of the present disclosure can have other capabilities not specifically described here. Further, while server system 1800 and client computing system 1814 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

Non-limiting example embodiments are provided here:

Embodiment A: A method comprising: retrieving, by a computing system comprising one or more processors and a memory with instructions executable by the one or more processors, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure; analyzing, by the computing system, the structured dataset and the unstructured dataset to generate a plurality of health indicators for the patient, wherein analyzing the structured dataset and the unstructured dataset comprises applying natural language processing to the free-form text in the report to extract one or more of the plurality of indicators; generating, by the computing system, based on the plurality of health indicators, one or more categorizations corresponding to the medical condition; determining, by the computing system, a treatment regimen based on drug orders in the structured dataset; performing, by the computing system, survival modeling to generate, by the computing system, based on (i) the plurality of health indicators, (ii) the one or more categorizations, and (iii) the treatment regimen, a prediction corresponding to a survival of the patient following administration of a treatment to the patient for the medical condition; and providing, by the computing system, a report comprising the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

Embodiment B: The method of Embodiment A, further comprising administering the treatment to the patient.

Embodiment C: The method of Embodiment A or B, wherein a treatment is administered only if the prediction indicates a likelihood of survival exceeding a threshold.

Embodiment D: The method of any of Embodiments A-C, further comprising determining that the prediction indicates a likelihood of survival exceeding a threshold, wherein the report comprises an indication of the likelihood of survival.

Embodiment E: The method of any of Embodiments A-D, wherein applying natural language processing to the free-form text comprises parsing the report using a plurality of expression patterns, each expression pattern comprising one or more operators.

Embodiment F: The method of any of Embodiments A-E, wherein one or more of the plurality of health indicators requires one or more of the expression patterns to be triggered.

Embodiment G: The method of any of Embodiments A-F, wherein the one or more categorizations comprise at least one of a cytogenetic category, a radiographic category, a molecular category, or a histological category.

Embodiment H: The method of any of Embodiments A-G, wherein the demographic and clinical data identifies a plurality of patient age, patient gender, the medical condition, or drugs administered to the patient.

Embodiment I: The method of any of Embodiments A-H, wherein the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

Embodiment J: The method of any of Embodiments A-I, wherein analyzing the structured dataset and the unstructured dataset further comprises generating tab-delimited tables based on the structured dataset.

Embodiment K: The method of any of Embodiments A-J, wherein generating the tab-delimited tables comprises extracting data from unmerged nested cells and reformatting tabs into the tab-delimited tables.

Embodiment L: The method of any of Embodiments A-K, wherein the medical condition is a cancer, and wherein the treatment is a cancer treatment.

Embodiment AA: A computing system comprising one or more processors and a memory with instructions configured to be executable by the one or more processors to cause the one or more processors to: retrieve, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure; analyze the structured dataset and the unstructured dataset to generate a plurality of health indicators for the patient, wherein analyzing the structured dataset and the unstructured dataset comprises applying natural language processing to the free-form text in the report to extract one or more of the plurality of indicators; generate, based on the plurality of health indicators, one or more categorizations corresponding to the medical condition; perform survival modeling to generate, based on the plurality of health indicators and the one or more categorizations, a prediction corresponding to a survival of the patient following administration of a treatment to the patient for the medical condition; and provide a report comprising one or more categorizations and/or the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

Embodiment BB: The system of Embodiment AA, wherein the instructions further cause the one or more processors to determine that the prediction indicates a likelihood of survival exceeding a threshold, wherein the report further includes an indication of the likelihood of survival.

Embodiment CC: The system of either Embodiment AA or BB, wherein applying natural language processing to the free-form text comprises parsing the report using a plurality of expression patterns, each expression pattern comprising one or more operators, wherein one or more of the plurality of health indicators requires one or more of the expression patterns to be triggered.

Embodiment DD: The system of any of Embodiments AA-CC, wherein the one or more categorizations comprise at least one of a cytogenetic category, a radiographic category, a molecular category, or a histological category.

Embodiment EE: The system of any of Embodiments AA-DD, wherein the demographic and clinical data identifies a plurality of patient age, patient gender, the medical condition, or drugs administered to the patient.

Embodiment FF: The system of any of Embodiments AA-EE, wherein the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

Embodiment GG: The system of any of Embodiments AA-FF, wherein analyzing the structured dataset and the unstructured dataset further comprises generating tab-delimited tables based on the structured dataset.

Embodiment HH: The system of any of Embodiments AA-GG, wherein generating the tab-delimited tables comprises extracting data from unmerged nested cells and reformatting tabs into the tab-delimited tables.

Embodiment II: The system of any of Embodiments AA-HH, further comprising performing tumor segmentation to identify a tumor region of interest (ROI) based on the MRI data prior to determining the tissue properties.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

Additional background and supporting information can be found in the following document(s), each of which is herein incorporated by reference:
Glass, J. L., D. Hassane, B. J. Wouters, H. Kunimoto, R. Avellino, F. E. Garrett-Bakelman, O. A. Guryanova, R. Bowman, S. Redlich, A. M. Intlekofer, C. Meydan, T. Qin, M. Fall, A. Alonso, M. L. Guzman, P. J. M. Valk, C. B. Thompson, R. Levine, O. Elemento, R. Delwel, A. Melnick and M. E. Figueroa (2017). “Epigenetic Identity in AML Depends on Disruption of Nonpromoter Regulatory Elements and Is Affected by Antagonistic Effects of Mutations in Epigenetic Modifiers.” Cancer Discov 7(8): 868-883.

What is claimed is:

1. A method comprising:
retrieving, by a computing system comprising one or more processors and a memory with instructions executable by the one or more processors, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure;
analyzing, by the computing system, the structured dataset and the unstructured dataset to generate a plurality of health indicators for the patient, wherein analyzing the structured dataset and the unstructured dataset comprises applying natural language processing to the free-form text in the report to extract one or more of the plurality of indicators;
generating, by the computing system, based on the plurality of health indicators, one or more categorizations corresponding to the medical condition;
determining, by the computing system, a treatment regimen based on drug orders in the structured dataset;
performing, by the computing system, survival modeling to generate, by the computing system, based on (i) the plurality of health indicators, (ii) the one or more categorizations, and (iii) the treatment regimen, a prediction corresponding to a survival of the patient following administration of a treatment to the patient for the medical condition; and
providing, by the computing system, a report comprising the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

2. The method of claim 1, further comprising administering the treatment to the patient.

3. The method of claim 2, wherein the treatment is administered only if the prediction indicates a likelihood of survival exceeding a threshold.

4. The method of claim 1, further comprising determining that the prediction indicates a likelihood of survival exceeding a threshold, wherein the report comprises an indication of the likelihood of survival.

5. The method of claim 1, wherein applying natural language processing to the free-form text comprises parsing the report using a plurality of expression patterns, each expression pattern comprising one or more operators.

6. The method of claim 5, wherein one or more of the plurality of health indicators requires one or more of the expression patterns to be triggered.

7. The method of claim 1, wherein the one or more categorizations comprise at least one of a cytogenetic category, a radiographic category, a molecular category, or a histological category.

8. The method of claim 1, wherein the demographic and clinical data identifies a plurality of patient age, patient gender, the medical condition, or drugs administered to the patient.

9. The method of claim 1, wherein the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

10. The method of claim 1, wherein analyzing the structured dataset and the unstructured dataset further comprises generating tab-delimited tables based on the structured dataset.

11. The method of claim 10, wherein generating the tab-delimited tables comprises extracting data from unmerged nested cells and reformatting tabs into the tab-delimited tables.

12. The method of claim 1, wherein the medical condition is a cancer, and wherein the treatment is a cancer treatment.

13. A computing system comprising one or more processors and a memory with instructions configured to be executable by the one or more processors to cause the one or more processors to:
retrieve, from an electronic health records (EHR) system, for a patient with a medical condition, a structured dataset and an unstructured dataset, the structured dataset comprising demographic and clinical data for the patient, and the unstructured dataset comprising a report with free-form text of a clinician with respect to a medical procedure;
analyze the structured dataset and the unstructured dataset to generate a plurality of health indicators for the patient, wherein analyzing the structured dataset and the unstructured dataset comprises applying natural language processing to the free-form text in the report to extract one or more of the plurality of indicators;
generate, based on the plurality of health indicators, one or more categorizations corresponding to the medical condition;
perform survival modeling to generate, based on the plurality of health indicators and the one or more categorizations, a prediction corresponding to a survival of the patient following administration of a treatment to the patient for the medical condition; and
provide a report comprising one or more categorizations and/or the prediction to one or more users for determining whether to administer the treatment to the patient for the medical condition, wherein providing the report comprises at least one of (1) transmitting the report to a computing device, (2) displaying the report on a display screen, or (3) storing the report in a non-volatile computer-readable storage medium for access via a server.

14. The system of claim 13, wherein the instructions further cause the one or more processors to determine that the prediction indicates a likelihood of survival exceeding a threshold, wherein the report further includes an indication of the likelihood of survival.

15. The system of claim 13, wherein applying natural language processing to the free-form text comprises parsing the report using a plurality of expression patterns, each expression pattern comprising one or more operators, wherein one or more of the plurality of health indicators requires one or more of the expression patterns to be triggered.

16. The system of claim 13, wherein the one or more categorizations comprise at least one of a cytogenetic category, a radiographic category, a molecular category, or a histological category.

17. The system of claim 13, wherein the demographic and clinical data identifies a plurality of patient age, patient gender, the medical condition, or drugs administered to the patient.

18. The system of claim 1, wherein the one or more health indicators corresponds to results of flow cytometry, cytogenetic assessment, fluorescence in-situ hybridization (FISH), a single nucleotide polymorphism (SNP) array, next generation sequencing (NGS) testing for gene mutations and/or rearrangements, and/or targeted molecular assays.

19. The system of claim 13, wherein analyzing the structured dataset and the unstructured dataset further comprises generating tab-delimited tables based on the structured dataset.

20. The system of claim 19, wherein generating the tab-delimited tables comprises extracting data from unmerged nested cells and reformatting tabs into the tab-delimited tables.
